(19) United States
(12) Patent Application Publication
(10) Pub. No.: US 2017/0247409 A1
(43) Pub. Date: Aug. 31, 2017

(54) ABIETANE-TYPE DITERPENOIDS

(71) Applicant: UNIVERSITY OF HELSINKI, University of Helsinki (FI)

(72) Inventors: Vãnia M. MOREIRA, Helsinki (FI); Adyary FALLARERO, Porvoo (FI); Jari YLI-KAUHALUOMA, Helsinki (FI); Pia VUORELA, Porvoo (FI); Mikko VAHERMO, Helsinki (FI)

(73) Assignee: UNIVERSITY OF HELSINKI, University of Helsinki (FI)

(21) Appl. No.: 15/516,142

(22) PCT Filed: Jul. 9, 2015
(86) PCT No.: PCT/FI2015/050495

§ 371 Date: Mar. 31, 2017

(30) Foreign Application Priority Data
Oct. 2, 2014 (FI) 20145858

(51) Int. Cl. C07K 5/083; A01N 37/46; A61K 31/223; C07C 323/59; C07D 209/20; A61K 31/405; A01N 43/38; C07K 5/062; A61K 31/198; C07C 235/82; C07D 213/55; A61K 31/4406; A01N 43/40; C07D 213/89; A61K 31/4425; C07C 251/44; A61K 45/06; C09D 5/14; C07C 233/63

(52) U.S. Cl. CPC C07K 5/0806; C07C 233/63; A01N 37/46; A61K 31/223; C07C 323/59; C07D 209/20; A61K 31/405; A01N 43/38; C07K 5/06026; A61K 31/198; C07C 235/82; C07D 213/55; A61K 31/4406; A01N 43/40; C07D 213/89; A61K 31/4425; C07C 251/44; A61K 45/06; C09D 5/14; C07C 2603/26; A61K 38/00

(57) ABSTRACT

The present invention relates to the field of wood rosin and resin acid derivatives and more particularly to abietane-type diterpenoids as well as different uses thereof. Furthermore, the present invention relates to methods of coating surfaces, preventing, reducing or inhibiting bacterial biofilm formation, and treating or preventing disorders caused by microbial growth and viability as well as bacterial colonization.

### FIELD OF THE INVENTION

The present invention relates to the field of wood rosin and resin acid derivatives and more particularly to abietane-type diterpenoids as well as different uses thereof. Furthermore, the present invention relates to methods of coating surfaces, preventing, reducing or inhibiting bacterial biofilm formation, and treating or preventing disorders caused by microbial growth and viability as well as bacterial colonization.

### BACKGROUND OF THE INVENTION

Rosin is rich in resin acids which have important ecological roles in host defense and promiscuous biological activities. Coniferous trees use oleo-resin (a complex mixture of mono-, sesqui-, and diterpenoids) for protection against foreign threats such as bark beetles and their vectored fungal pathogens (oleorosin accumulates at the wound site to kill invaders and both flush and seal the injury), and also as a source of major biosynthetic building blocks.

The solid portion of the resin that can be obtained by evaporation of its volatiles by heating is named rosin, and is mainly comprised of abietic and dehydroabietic acids, (1*) and (1) respectively, two diterpenoids usually known as resin acids.

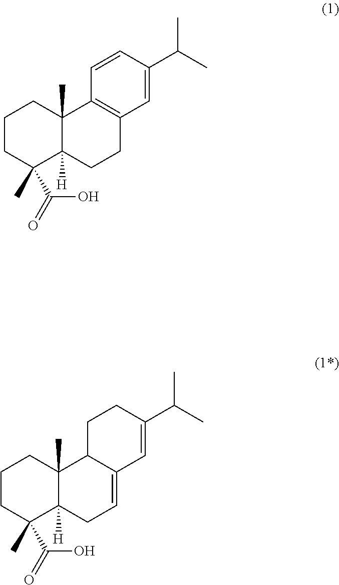 [SMILES: CC(C)c1ccc2c(c1)CC[C@H]1[C@](C)(C(=O)O)CCC[C@]21C ; CC(C)C1=CC2=CC[C@H]3[C@](C)(C(=O)O)CCC[C@]3(C)C2CC1]

The physico-chemical properties of rosin make it appealing for inclusion in soldering fluxes, varnishes, antifouling paints, soaps and glues. Rosin is also used in the pharmaceutical industry as a glazing agent for the manufacture of medicines. More recently, the use of the resin-based products has come into the spotlight and for example formulations containing resin salve or lacquer have become commercially available for use as human health products for wound-healing and treatment of nail fungal infections.

The bioactive properties of resin-based products are due to the presence of the resin acids and relate to their ecological role in host defence. Nonetheless, despite their beneficial effects, resin acids are promiscuous in their biological activities which include antitumor, antimicrobial, anti-leishmanial, and anti-malarial activities among others, and like many other natural products, are not fully optimized for the treatment of human diseases. Moreover, they are contact allergens and allergic reactions to resin-based products are frequent side effects. Resin acids are however excellent starting materials that can be chemically modified to produce more potent and selective compounds targeting specific biological activities.

Amino acids are indispensable in nature as building blocks for peptide and protein synthesis in higher organisms. Due to their ability to influence vital biological processes in different organisms, amino acids have been traditionally regarded as “privileged moieties” in drug discovery programs. For simple unicellular organisms, amino acids are vital in mediating signal transduction processes and as nutrients to support growth. For instance, bacteria use amino acids as nutrients to support bacterial growth, to regulate bacterial spore germination, and as components of the cell wall (Kolodkin-Gal et al. 2010; Hochbaum et al. 2011).

Bacteria can switch into two different life-styles: single-cells (plank-tonic mode; suspended) and multi-cellular (biofilms mode; embedded in a biopolymer matrix of complex composition). Single-cells have long been connected with acute infections, which are generally treatable with antibiotics, provided that an accurate and fast diagnosis is made, which is also generally possible. However, if planktonic bacteria switch into biofilms in the human host, a chronic infection will occur and can become completely incurable. Biofilms represent the actual bacterial lifestyle outside laboratory conditions. They are dramatically widespread and significantly impact both economy and human health (Donlan and Costerton, 2002).

From the human health perspective, over 65% of bacterial infections are nowadays recognized as biofilms-related, i.e. lung pneumonia of cystic fibrosis (CF) patients, otitis media, chronic wounds, Legionnaire's disease and nosocomial infections which have risen due to an increased use of medical devices (i.e. prosthetic implants, catheters, pacemakers, wound dressings and contact lenses) (Coenye et al. 2014). Hospital-acquired infections in the US nowadays have been recognized to cause more annual deaths than emphysema, AIDS, Parkinson's disease and homicides combined, with S. aureus being among the most common responsible pathogens (Worthington et al. 2013). In many cases, for instance when biofilms are restricted to a certain localized tissue, the best available solution is the surface “clean-up” and biofilm removal by surgical means. Consequently, that increases the need for longer hospitalization, boosting the re-infection chances.

So far, still a limited repertoire of easily available compounds has been reported that can selectively act in vitro and eradicate existing biofilms at low concentrations, especially in the case of those formed by S. aureus (Landini et al. 2010; Blackledge et al 2013). To date, one disinfectant (commercially sold by Sterilex, USA) has been approved by a regulatory agency to be used specifically against bacterial biofilms. However, no antibiotic has been approved yet as an anti-biofilm agent. Furthermore, lack of potent controls is also a common scenario in basic biofilm studies, since even millimolar concentrations of antibiotics are not enough to cause high inhibitory effects (Tote et al, 2009; Skogman et al, 2012).

A few diterpenoids have so far been reported to bear anti-biofilm activity. Salvipisone, a naturally occurring diterpenoid available only in very limited amounts, was reported to prevent the adhesion of S. aureus (ATCC 29213) at high micromolar concentrations, which coincided with its bacterio-static effects, measured by minimum inhibitory concentration (MIC) values (Kuzma et al. 2007). 4-Epi-pimaric acid prevents biofilm formation by the oral pathogen S. mutans at micromolar concentrations but no activity on existing biofilms has been reported (Ali et al. 2012). Rubesanolide D inhibited biofilm formation of the dental bacterium S. mutans (Zou et al. 2012). Also, the anti-biofilm activity of abietic acid has been reported (Tsuchiya et al. 2010). We previously reported that nordehydroabietylamine, dehydroabietic acid, and dehydroabietylamine prevented S. aureus biofilm formation in the low micromolar range, and unlike typical antibiotics, only 2 to 4-fold higher concentrations were needed to significantly reduce viability and biomass of existing biofilms (Fallarero et al, 2013). Dehydroabietic acid 1 was the most selective towards biofilm bacteria, achieving high killing efficacy and it was best tolerated by three different mammalian cell lines.

From a drug discovery perspective, there is still a very limited selection of molecules (even in the pre-clinical stage) that can selectively act for example in vitro on biofilms and eradicate them at low concentrations. Thus, there is a dramatic need for new, improved, and cost-effective anti-biofilm solutions.

### BRIEF DESCRIPTION OF THE INVENTION

An object of the present invention is to provide novel molecules with excellent anti-biofilm and/or anti-microbial properties as well as methods and uses related thereto. The present invention solves the above problems i.e. lack of effective and low-cost anti-microbial (e.g. anti-S. aureus) agents. The objects of the invention are achieved by methods and arrangements which are characterized by what is stated in the independent claims. The preferred embodiments of the invention are disclosed in the dependent claims.

The design of the compounds described in this invention explores the ecological roles of natural products to produce derivatives of resin acids with anti-biofilm activity. The present invention is based on the idea that the compounds utilize diterpenic resin acids and furthermore combine two active scaffolds via a simple chemical procedure, which results in a significant enhancement of anti-biofilm activity against a model clinical pathogen. These compounds exhibit potencies that are remarkably higher than those of reported anti-biofilm agents, and therefore they represent a chemical structure for new antibiotic types and other anti-biofilm products.

Indeed, the present invention is based on a novel type of abietane-type diterpenoids, wherein the uniqueness of the compounds relies on the biological activity that results from the combination of the abietane scaffold of diterpenic resin acids with amino acid (either, L-, D-, or unusual) and/or peptidic moieties, in a single molecule.

The parent resin acids suitable for preparing the compounds of the present invention include resin acids that are naturally occurring and highly abundant from the rosin of coniferous trees. Thus, the present invention also provides new uses for raw materials such as wood rosin, widely abundant and easily obtainable from coniferous forests (such as those predominant in Finland). Wood rosin is a low-value side stream of the forest industry, and places Finland in a privileged position for its global supply.

In addition, the preparation of these small molecular weight compounds relies on very simple, inexpensive and high yielding synthetic chemistry methods, thus affording the active compounds in high amounts and excellent purity, in only few (e.g. 2-3) reaction steps.

Also, the present invention enables effective chemical tools for application in translational anti-biofilm applications (dressings and chemotherapy formulations for wounds, bio-coatings of medical devices, bio-desinfectants, anti-fouling cleaning solutions, etc). By the present invention it is also possible to provide diterpenoid-based compounds, which may be utilized for example either as molecular probes or as leads for the development of new anti-biofilm agents and/or drugs.

According to our findings, the derivatives of the present invention prevent in vitro bacterial colonization and biofilm formation at low micromolar concentrations and, upon addition to existing S. aureus biofilms, they also significantly reduce the core of viable cells. This latter effect can be achieved at concentrations that are only 2 to 4-fold higher than the ones needed to inhibit biofilm formation, in contrast to currently available antibiotics that commonly require up to 1000-fold higher concentrations. Thus low dosages of the compound are enough to reach excellent therapeutic efficacy. Our mechanistic studies point out to the bacterial membrane as one (likely primary) target of the anti-biofilm effects of these derivatives, in a manner resembling the action mechanisms of antimicrobial peptides (AMPs). Our observations also support the fact that these compounds display potent antibacterial properties against the single-cell state, and are not exclusively acting on the biofilms lifestyle. Because bacteria are dynamically switching between single-cell and biofilm states in host organisms, and the predominant lifestyle can shift depending on various environmental factors, the multifunctional antimicrobial profile of these compounds makes them extremely advantageous in comparison to available antibiotics and/or biocides. The compounds described herein exhibit potencies that are remarkably high when compared to the known repertoire of active anti-biofilm compounds and they represent a highly feasible chemical foundation for new antibiotic types as well as other anti-biofilm products. Thus, the present invention solves the problems of conventional unsuccessful and unspecific therapies. Furthermore, the compounds of the present invention are safe (biocompatibility index (BI)) and thus less likely to cause side effects on a treated subject.

The present invention relates to a compound of formula (I) for use in treatment or prevention of bacterial biofilms and/or other microbial infections

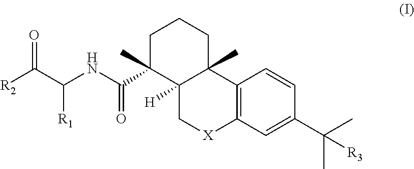

wherein

X is selected from CH₂, C═O and C═N—OH;

each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH, OR′ or an amino acid residue of formula —Y1 or a dipeptide residue of formula —Y1Y2 or a C₁₋₈-alkyl ester of said amino acid or said dipeptide residue; and

R3 is H, OOH, OOR′, or OH;

wherein

Y1 and Y2 are each independently selected from natural and non-natural amino acids comprising in its side chain 0 to 15 carbon atoms and optionally 1 to 4 heteroatoms;

R is H or C₁₋₃-alkyl; and

R′ is C₁₋₈-alkyl;

or pharmaceutically acceptable salt thereof.

The present invention also relates to novel compounds of formula (I)

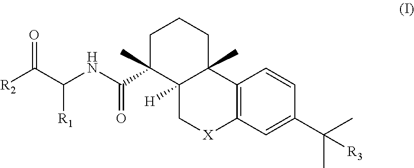

wherein

X is selected from CH₂, C═O and C═N—OH;

each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH, OR′ or an amino acid residue of formula —Y1 or a dipeptide residue of formula —Y1Y2 or a C₁₋₈-alkyl ester of said amino acid or said dipeptide residue; and

R3 is H, OOH, OOR′, or OH;

wherein

Y1 and Y2 are each independently selected from natural and non-natural amino acids comprising in its side chain 0 to 15 carbon atoms and optionally 1 to 4 heteroatoms;

R is H or C₁₋₃-alkyl; and

R′ is C₁₋₈-alkyl;

or a pharmaceutically acceptable salt thereof;

provided that when X is CH₂, R2 is OH, and R3 is H, R1 is not H, iso-propyl or benzyl, or when X is CH₂, R2 is OH, R3 is H, and R1 is in D-configuration, R1 is not isobutyl, p-OH substituted benzyl, indolyl or methyl-S-propanyl.

Furthermore, the present invention relates to the compound of the present invention for use as a medicament.

Furthermore, the present invention relates to the compound of the present invention for use in treatment or prevention of bacterial biofilms and/or other microbial infections.

Furthermore, the present invention relates to a method of coating a surface of a material, wherein said method comprises applying a composition comprising a compound of formula (I) of the present invention to the surface of the material.

Still, the present invention relates to a use of a compound of formula (I) of the present invention for coating a surface of a material.

Still, the present invention relates to a coating comprising a compound of formula (I) of the present invention.

Still, the present invention relates to a surface coated material, wherein the coating comprises a compound of formula (I) of the present invention.

Still, the present invention relates to a method of preventing, reducing or inhibiting bacterial biofilm or microbial formation, wherein said method comprises applying a composition comprising a compound of formula (I) of the present invention into a material or to a surface of a material.

Still, the present invention relates to a use of a compound of formula (I) of the present invention for preventing, reducing or inhibiting bacterial biofilm or microbial formation in or on a material.

Still, the present invention relates to a use of a compound of formula (I) of the present invention in medical devices, water filtration systems, ship hulls, textiles, furniture, food and food-related related surfaces, pharmaceuticals and devices for drug delivery, dressings, coatings, laboratory devices, biosensors, materials for patterned cell culture, diagnostic kits, cleaning solutions or desinfectants.

Still, the present invention relates to a method of treating or preventing disorders caused by microbial growth and viability as well as bacterial colonization in a subject, wherein said method comprises administering an effective amount of a composition comprising a compound of formula (I) of the present invention to the subject in need thereof.

Still, the present invention relates to a process for preparing the compound of formula (I) of the present invention, wherein said method comprises coupling of an amino acid or peptidic residue to the core of dehydroabietic acid in order to obtain the compound of formula (I) of the present invention.

Other objects, details and advantages of the present invention will become apparent from the following drawings, detailed description and examples.

### BRIEF DESCRIPTION OF THE DRAWINGS

In the following the invention will be described in greater detail by means of specific embodiments with reference to the attached drawings, in which

FIG. 1 shows time-course of S. aureus ATCC 25923 biofilms killing effects by compounds 22 and 25. Biofilms were formed during 18 hours (as in the pre-exposure assay) and compounds were then added. Effects on biofilms viability were measured during the first four hours up to 24 hours (similar conditions to the post-exposure assay); and

FIG. 2 shows studies supporting a putative membrane-targeting effect of compounds 22 and 25. A: quantification of ATP release from the S. aureus ATCC 25923 biofilms using the BacTiter-Glo bioluminescent assay. B: quantification of membrane depolarization in treated biofilms by the potential-sensitive DiABC(4)3 probe; this probe can only enter depolarized cells, where it experienced an enhanced fluorescence signal upon binding to intracellular proteins. For statistical comparisons between the treated and untreated samples, an unpaired t-test with Welch's correction was used, using GraphPad Prism (v 5.0 for Mac OS X).

### DETAILED DESCRIPTION OF THE INVENTION

The present invention relates to compounds that are remarkably active on chemotolerant micro-organisms (e.g. Staphylococcus aureus strains) and offer a new naturally-inspired anti-biofilm and/or anti-microbial chemotype significantly more potent than the currently available antibiotics. “Optional” or “optionally” as used herein or hereafter denotes that the subsequently described event or circumstance may but need not occur, and that the description includes instances where the event or circumstance occurs and instances in which it does not. The term “optionally substituted” as used herein and hereafter e.g. in context of a Cy group denotes cycloalkyl, (hetero)cyclyl or (hetero)aryl that is either un-substituted or substituted independently with one or more, in particular 1, 2, or 3, substituent(s) attached at any available atom to produce a stable compound, e.g. a phenyl group may be substituted once with a denoted substituent attached to o-, p- or m-position of the phenyl ring. In general “substituted” refers to a substituent group as defined herein in which one or more bonds to a hydrogen atom contained therein are replaced by a bond to a non-hydrogen atom unless otherwise denoted.

The term “comprise” as used herein and hereafter describes the constituents of the compositions of the present invention in a non-limiting manner i.e. the said composition comprising constituents consists of, at least, the said constituents, but may additionally, when desired, comprise other constituents. However, the said composition of the present invention comprising said constituents may consist of only the said constituents. The term “comprise” is further used to reflect that the composition of the present invention may comprise trace components of other materials or other impurities, or both, which do not alter the effectiveness or the safety of the mixture.

The expression “pharmaceutically acceptable” represents being useful in the preparation a pharmaceutical product or composition that is generally safe, non-toxic, and neither biologically nor otherwise undesirable, and includes being useful for both veterinary use as well as human pharmaceutical use. The expression “pharmaceutically acceptable salt” includes any non-toxic organic and inorganic acid or base addition salts that compounds of formula (I) can form. Said salts are known to a person skilled in the art.

### Compounds

The compounds of the present invention are ((1R,4aS,10aR)-7-isopropyl-1,4a-dimethyl-1,2,3,4,4a, 9,10,10a-octahydrophenanthrene-1-carbon-yl) derivatives i.e. N-abiet-8,11,13-trien-18-oyl derivatives further comprising an amino acid side chain or a short peptide side chain coupled to the dehydroabietic acid core. The end group of the amino acid or peptide side chain may also exist as the corresponding alkyl ester.

The term “halogen” as used herein and hereafter by itself or as part of other groups refers to the Group VIIa elements and includes F, Cl, Br and I groups.

The term “alkyl” as used herein and hereafter as such or as part of haloalkyl, perhaloalkyl or alkoxy group is an aliphatic linear, branched or cyclic, especially linear or branched, hydrocarbon group having the indicated number of carbon atoms, for example C₁₋₆-alkyl has 1 to 6 carbon atoms in the alkyl moiety and thus, for example, C₁₋₄-alkyl includes methyl, ethyl, n-propyl, isopropyl, n-butyl, sec-butyl, isobutyl, tert-butyl and C₁₋₆-alkyl additionally includes branched and straight chain pentyl and hexyl.

The term “haloalkyl” as used herein and hereafter refers to any of the above alkyl groups where one or more hydrogen atoms are replaced by halogen(s): in particular I, Br, F or Cl. Examples of haloalkyl groups include without limitation chloromethyl, fluoromethyl and —CH₂CF₃. The term “perhaloalkyl” is understood to refer to an alkyl group, in which all the hydrogen atoms are replaced by halogen atoms. Preferred examples include trifluoromethyl (—CF₃) and trichloromethyl (—CCl₃).

The term “C₃₋₆-cycloalkyl” as used herein and hereafter refers to cycloalkyl groups having 3 to 6 carbon atoms and thus includes cyclopropyl, cyclobutyl, cyclopentyl, and cyclohexyl.

The term “alkylenyl” as used herein and hereafter, is a divalent group derived from a straight or branched chain hydrocarbon of having suitably 1 to 6 carbon atoms. Representative examples of alkylenyl include, but are not limited to, —CH₂—, —CH(CH₃)—, —C(CH₃)₂—, —CH₂CH₂—, —CH₂CH₂CH₂—, —CH₂CH₂CH₂CH₂—, and —CH₂CH(CH₃)CH₂—.

The term “C₁₋₆-alkoxy” as used herein and hereafter refers to a —O—(C₁₋₆-alkyl) group where the “C₁₋₆-alkyl” has the above-defined meaning. Examples of preferred alkoxy groups include, but are not limited to, methoxy, ethoxy, and isopropyloxy.

Thus the present invention provides compound of formula (I) for use in treatment or prevention of bacterial biofilms and other microbial infections

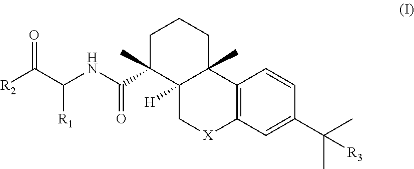

wherein

X is selected from CH₂, C═O and C═O and C═N—OH;

each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH, OR′ or an amino acid residue of formula —Y1 or a dipeptide residue of formula —Y1Y2 or a C₁₋₆-alkyl ester of said amino acid or said dipeptide residue; and

R3 is H, OOH, OOR′, or OH;

wherein

Y1 and Y2 are each independently selected from natural and non-natural amino acids comprising in its side chain 0 to 15 carbon atoms and optionally 1 to 4 heteroatoms;

R is H or C₁₋₃-alkyl; and

R′ is C₁₋₆-alkyl;

or pharmaceutically acceptable salt thereof.

The present invention further provides novel compounds of formula

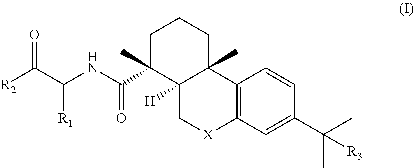

wherein

X is selected from CH₂, C═O and C═N—OH;

each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH, OR′ or an amino acid residue of formula —Y1 or a dipeptide residue of formula —Y1Y2 or a C₁₋₆-alkyl ester of said amino acid or said dipeptide residue; and

R3 is H, OOH, OOR′, or OH;

wherein

Y1 and Y2 are each independently selected from natural and non-natural amino acids comprising in its side chain 0 to 15 carbon atoms and optionally 1 to 4 heteroatoms;

R is H or C₁₋₃-alkyl; and

R′ is C₁₋₆-alkyl;

or a pharmaceutically acceptable salt thereof;

provided that when X is CH₂, R2 is OH, and R3 is H, R1 is not H, iso-propyl or benzyl, or when X is CH₂, R2 is OH, R3 is H, and R1 is in D-configuration, R1 is not isobutyl, p-OH substituted benzyl, indolyl or methyl-S-propanyl.

The present invention still further provides novel compounds of formula of formula (I)

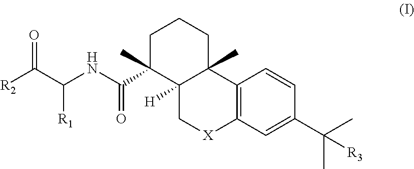

wherein

X is selected from CH₂, C═O and C═N—OH;

each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH, OR′ or an amino acid residue of formula —Y1 or a dipeptide residue of formula —Y1Y2 or a C₁₋₆-alkyl ester of said amino acid or said dipeptide residue; and

R3 is H, OOH, COOR′, or OH;

wherein

Y1 and Y2 are each independently selected from natural and non-natural amino acids comprising in its side chain 0 to 15 carbon atoms and optionally 1 to 4 heteroatoms;

R is H or C₁₋₃-alkyl; and

R′ is C₁₋₆-alkyl;

or a pharmaceutically acceptable salt thereof;

provided that

when X is CH₂, R2 is OH, and R3 is H, R1 is not H, Me, L—CH(CH₃)₂, CH₂OH, L—CH₂Ph, L-indolyl, —(CH₂)COOH; L—(CH₂)₂COOH; (CH₂)₂SMe; or

when X is CH₂, R2 is OMe, and R3 is H, R1 is not H, L—Me, L—CH₂COOMe, L—CH(CH₃)CH₂CH₃, L—CH₂CH(CH₃)₂, CH₂Ph, L—CH₂OH, L—CH₂(C₆H₄)-p-OH or L—CH(CH3)₂.

In an aspect of the present invention X is CH₂ or C═O, preferably CH₂.

Preferably each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is cyclohexyl, phenyl, pyridynyl, or indolyl, any of which may be optionally substituted.

Even more preferably each R1 is CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; in particular Cy is cyclohexyl, phenyl, pyridynyl, or indolyl, any of which may be optionally substituted as indicated.

In an aspect of the present invention R1 is selected from the group consisting of —H, —CH(CH₃)₂, —CH₂CH₃, CH₂CH(CH₃)₂, CH₂CH₂SCH₃,

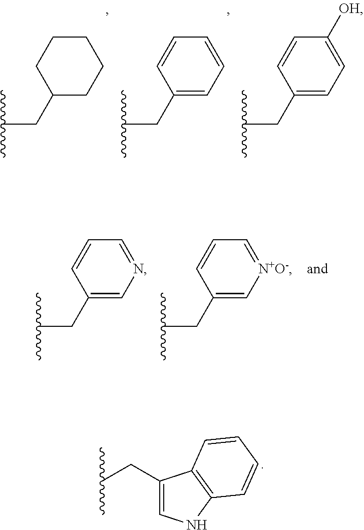

In a particularly preferred aspect of the present invention R1 is selected from the group consisting of:

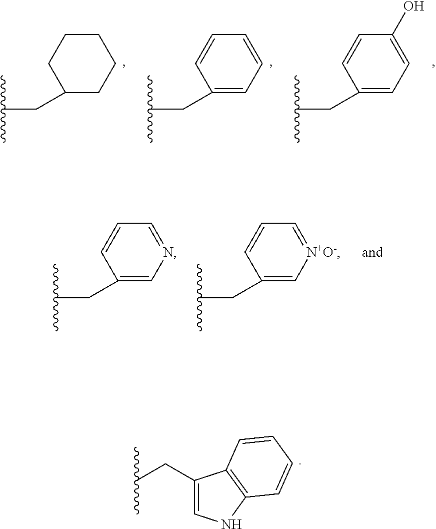

Y1 and Y2 are each independently preferably selected from known natural and non-natural amino acids such as those listed in Wagner, Ingrid; Musso, Hans (November 1983). “New Naturally Occurring Amino Acids”. Angew. Chem. Int. Ed. Engl. 22 (22): 816-828. In accordance with the present invention the said amino acid residue may exist in either L- or D-configuration.

In particular Y1 and Y2 are each independently selected from histidine, alanine, isoleucine, arginine, leucine, asparagine, lysine, aspartic acid, methionine, cysteine, phenylalanine, cyclohexylalanine, glutamic acid, threonine, glutamine, tryptophan, glycine, valine, ornithine, serine and tyrosine.

In an aspect of the present invention Y1 and Y2 are each independently selected from glycine, valine, leucine, phenylalanine, cyclohexylalanine, methionine, tyrosine, and tryptophane.

In one aspect of the present invention R2 is OH or OR′. Preferably R′ is methyl or ethyl. In a preferred aspect of the invention R2 is OH. The resulting free carboxyl group is particularly beneficial for the anti-biofilm activity of the present compounds.

In a preferred aspect of the invention R2 is OH and R1 is CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; in particular Cy is cyclohexyl, phenyl, pyridynyl, or indolyl, any of which may be optionally substituted as indicated; in particular R1 is selected from:

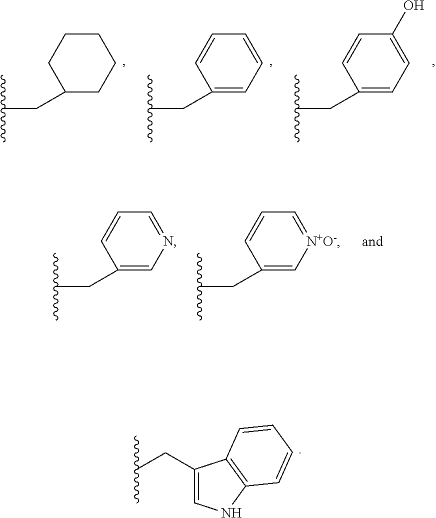

In alternative aspect of the present invention R2 is an amino acid residue of formula —Y1 or a C₁₋₆-alkyl ester of said amino acid residue. In a preferred embodiment of this aspect of the present invention Y1 is selected from histidine, alanine, isoleucine, arginine, leucine, asparagine, lysine, aspartic acid, methionine, cysteine, phenylalanine, cyclohexylalanine, glutamic acid, threonine, glutamine, tryptophan, glycine, valine, ornithine, serine and tyrosine, more preferably from glycine, valine, leucine, phenylalanine, cyclohexylalanine, methionine, tyrosine, and tryptophane.

In still another alternative aspect of the present invention R2 is a dipeptide residue of formula —Y1Y2 or a C₁₋₆-alkyl ester of said dipeptide residue. In a preferred embodiment of this aspect of the present invention Y1 and Y2 are each independently is selected from histidine, alanine, isoleucine, arginine, leucine, asparagine, lysine, aspartic acid, methionine, cysteine, phenylalanine, cyclohexylalanine, glutamic acid, threonine, glutamine, tryptophan, glycine, valine, ornithine, serine and tyrosine, more preferably from glycine, valine, leucine, phenylalanine, cyclohexylalanine, methionine, tyrosine, and tryptophane.

In a particular aspect of the present invention provided is a compound of formula (I),

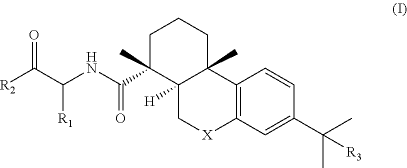

wherein

X is selected from CH₂ and C═O;

R1 is CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and

R2 is OH; and

R3 is H, OOH, COOR′, or OH;

wherein

R is H or C₁₋₃-alkyl; and

R′ is C₁₋₆-alkyl;

or a pharmaceutically acceptable salt thereof.

Preferably present compounds are for use in treatment or prevention of a disease or a condition involving or resulting from bacterial biofilms and/or other microbial infections or treatment or prevention of disorders caused by microbial growth and viability as well as bacterial colonization in a subject, in particular wherein treatment or prevention of a disease or a condition is reached by achieving a level of antibacterial or antimicrobial activity sufficient to inhibit bacteria or microbes, or the growth, viability or colonization thereof.

In an aspect of the present invention the invention relates to a compound of formula (I) selected from the group consisting of:

Methyl N-(abiet-8,11,13-trien-18-oyl) glycinate (3);

Methyl N-(abiet-8,11,13-trien-18-oyl) L-valinate (4);

Methyl N-(abiet-8,11,13-trien-18-oyl) L-ethylglycinate (5);

Methyl N-(abiet-8,11,13-trien-18-oyl) L-leucinate (6);

Methyl N-(abiet-8,11,13-trien-18-oyl) L-phenylalaninate (7);

Methyl N-(abiet-8,11,13-trien-18-oyl) cyclohexyl-L-alaninate (8);

Methyl N-(abiet-8,11,13-trien-18-oyl) D-methioninate (9);

Methyl N-(abiet-8,11,13-trien-18-oyl) D-tyrosinate (10);

Methyl N-(abiet-8,11,13-trien-18-oyl) D-tryptophanate (11);

Ethyl N-(abiet-8,11,13-trien-18-oyl) glycyl-glycinate (14);

Methyl N-(abiet-8,11,13-trien-18-oyl) L-alanyl-L-alanyl-L-alaninate (16);

N-(abiet-8,11,13-trien-18-oyl) glycine (17);

N-(abiet-8,11,13-trien-18-oyl) L-valine (18);

N-(Abiet-8,11,13-trien-18-oyl) L-ethylglycine (19);

N-(Abiet-8,11,13-trien-18-oyl) L-leucine (20);

N-(Abiet-8,11,13-trien-18-oyl) L-phenylalanine (21);

N-(Abiet-8,11,13-trien-18-oyl) cyclohexyl-L-alanine (22);

N-(Abiet-8,11,13-trien-18-oyl) D-methionine (23);

N-(abiet-8,11,13-trien-18-oyl) D-tyrosine (24);

N-(Abiet-8,11,13-trien-18-oyl) D-tryptophan (25);

N-(Abiet-8,11,13-trien-18-oyl) glycyl-glycine (27);

Methyl N-(7-oxo-abiet-8,11,13-trien-18-oyl) glycinate (28);

Methyl N-(7-oxo-abiet-8,11,13-trien-18-oyl) cyclohexyl-L-alaninate (29);

Methyl N-(abiet-8,11,13-trien-18-oyl) D-phenylalaninate (30);

N-(Abiet-8,11,13-trien-18-oyl) D-phenylalanine (31);

N-(7-Oxoabiet-8,11,13-trien-18-oyl) cyclohexyl-L-alanine (32);

Methyl N-(abiet-8,11,13-trien-18-oyl) H-(3 (3-pyridyl)-D-alaninate (33);

Methyl N-(abiet-8,11,13-trien-18-oyl) H-6-(3-pyridyl-N-oxide)-D-alaninate (34);

Methyl N-(abiet-8,11,13-trien-18-oyl) H-6-(3-pyridyl)-D-alanine (35);

Methyl N-(7-hydroxyiminoabiet-8,11,13-trien-18-oyl) cyclohexyl-L-alaninate (36);

and pharmaceutically acceptable salts thereof.

The compounds of the present invention can be prepared by method known to a person skilled in the art. For example the compounds of the present invention can be prepared by the following reaction sequences:

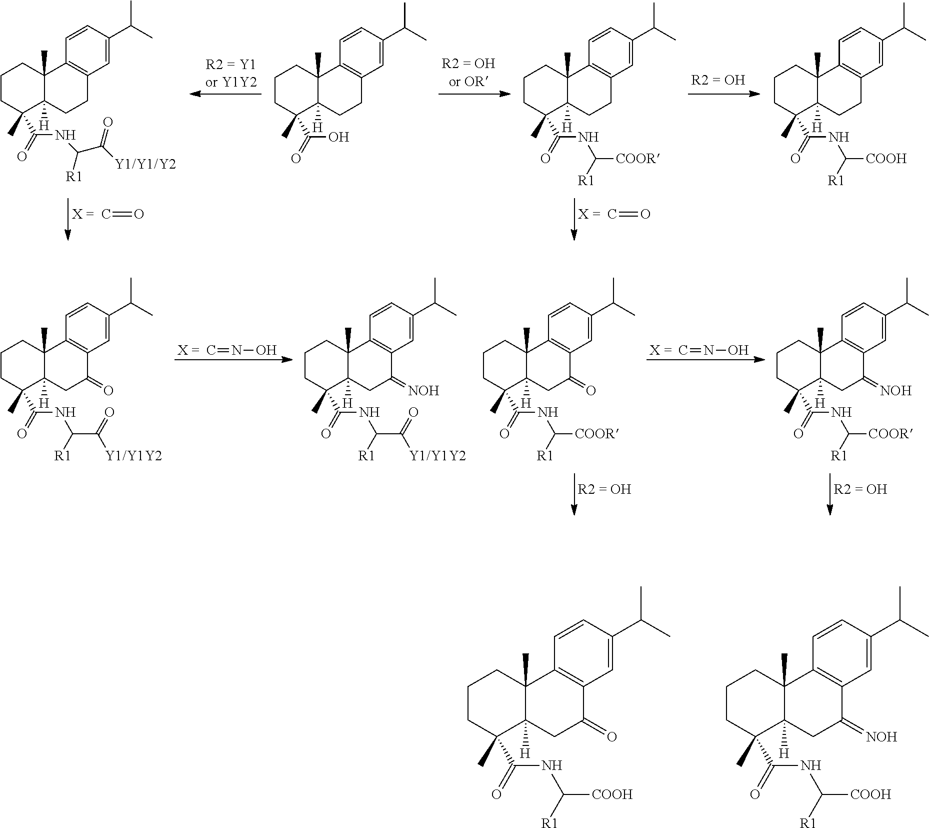

A process for preparing novel compounds of formula (I) of the present invention comprises coupling of an amino acid residue or a peptidic residue to dehydroabietic acid in order to said compounds of formula (I).

### Pharmaceutical Compositions and Administration

In one aspect of the invention compositions comprising the compound of the present invention may be used for medical purposes i.e. treating or preventing microbial infections and/or bacterial biofilms or disorders caused by microbial growth and viability as well as bacterial colonization.

As used herein, the term “treatment” or “treating” refers to administration of a composition comprising a compound of formula (I) of the present invention in an effective amount to a subject for purposes which include not only complete cure but also amelioration or alleviation of disorders or symptoms related to a microbial infection, bacterial biofilm or microbial growth and viability as well as bacterial colonization in question. “Treatment” or “treating” may also refer to any reduction in the number or viability of bacteria or microbes, or to slowing down the growth or colonization of bacteria or microbes. Therefore, “effective amount” or “therapeutically effective amount” refers to an amount with which the number or viability of bacteria or microbes is reduced, the growth or colonization of bacteria or microbes is slowed down or the harmful effects of a microbial infection in question are, at a minimum, ameliorated. In a specific embodiment of the invention the growth, viability or colonization of bacteria is inhibited or reduced. In this case the harmful effects include but are not limited to itch, pain, coughing and sneezing, fever, septicemia, pneumonia, inflammation, vomiting, diarrhea, fatigue, tissue damage and cramping. The harmful effects may be caused by the immune system of the host, which tries to clear the infectious organisms (e.g. inflammation) from the human organism, or by the micro-organism itself (e.g. tissue damage).

“Therapeutic effectiveness” may be based on either in vitro results or clinical outcome, and does not require that a compound of the present invention kills 100% of the bacteria or microbes involved in an infection. Successful treatment depends on achieving a level of antibacterial or antimicrobial activity sufficient to inhibit bacteria or microbes, or the growth, viability or colonization thereof. The effects of the compound of formula (I) may be either short term or long term. The effect of the compounds of the present invention may be studied in a variety of in vivo settings or in vitro tests, which for example relate to determinations of the MIC or minimum bactericidal concentration (MBC) of an agent (see e.g. examples of the present disclosure). Examples of the present disclosure describe several suitable methods for testing the effect of a compound. Suitable settings and tests are well known to a person skilled in the art.

Microbes can cause acute infections, chronic infections, which can last for weeks, months, or a lifetime, or latent infections, which may not cause symptoms at first but can reactivate over a period of months or years. Bacterial or microbial infections can cause mild, moderate, and severe diseases. As used herein “microbial infections” refers to invasion of a host organism's body tissues by microbes, their multiplication, and the reaction of host tissues to these organisms and the toxins they produce. “Microbes” refer to microorganisms, i.e. microscopic organisms, which may be single cell or multicellular organisms. Microorganisms include but are not limited to all the bacteria and archaea, and some protozoa, fungi and algae. In a specific embodiment of the invention bacteria are Gram-positive bacteria, Gram-negative bacteria, planktonic bacteria, bacteria growing in a biofilm or any combination thereof. In another specific embodiment of the invention the bacteria are selected from the group consisting of various strains of planktonic bacteria, Staphylococcus spp. including Staphylococcus aureus and Staphylococcus epidermidis, Escherichia coli or any combination thereof.

As used herein “bacterial biofilms” refers to an organized and well-structured community of bacterial cells embedded within a self-produced matrix of extracellular polymeric substance that may or not be attached to a surface. In contrast to biofilms, planktonic cells of the same organism are single-cells that may float or swim in a liquid medium. Biofilms may form on living or non-living surfaces. Biofilm growth may occur for example in teeth, heart valves (endocarditis), lungs of cystic fibrosis patients causing chronic bronchopneumonia, middle ear in patients with chronic and secretory otitis media, intravenous catheters and stents and chronic wounds, and it may cause chronic infections, persisting inflammation or tissue damage.

The bacterial or microbial infections to be treated according to the present invention include for example bacteremia, septicemia, skin and soft tissue infection, bacterial tissue damage, impetigo, lung pneumonia of cystic fibrosis patients, meningitis, otitis media, rhinosinusitis, chronic osteomyelitis, chronic wounds, Legionnaire's disease, infections in the pelveoperitoneal region, fever in hematological patient, infection associated with an intravenous line or other catheter, canyl and/or device, prosthetic joint infections, infection in gastrointestinal tract, in the eye, or in the ear, superficial skin infection, and colonization of gastrointestinal tract, mucous membranes and/or skin by noxious bacteria. The bacterial infectious diseases include, but are not limited to, severe hospital-acquired infections, infections of the immunocompromised patients, infections of the organ transplant patients, infections at the intensive care units (ICU), severe infections of wounds, in particular of burn wounds, severe community-acquired infections as well as infections caused by multi-resistant bacteria. In a specific embodiment of the invention the disorder caused by bacteria is selected from the group consisting of bacterial infections, inflammation caused by bacteria, bacterial tissue damage, lung pneumonia of cystic fibrosis patients, otitis media, chronic wounds, Legionnaire's disease, nosocomial infections and hospital-acquired infections such as those arising from the use of indwelling medical devices.

In humans, the antibiotic tolerance of biofilm communities hampers the treatment of persistent bacterial infections and chronic wounds. MIC and MBC of antibiotics to biofilm growing bacteria may be up to 100-1 000 fold higher than that of planktonic bacteria. Indeed, the currently available antibiotics are ineffective on bacterial biofilms even in high milimolar concentrations.

According to the present invention one, two or several compounds of the present invention (either having same or different formulas) may be administered to a subject in a pharmaceutical composition. A pharmaceutical composition comprises at least one compound of the invention, their pro-drug or salt forms or selected combinations thereof. In addition a pharmaceutical composition may also comprise any other therapeutically effective agents, any other agents, such as a pharmaceutically acceptable solvent, diluent, carrier, buffer, excipient, adjuvant, antiseptic, or filling, stabilizing, thickening, wetting, dispersing, solubilizing, suspending, emulsifying, binding, disintegrating, encapsulating, coating, embedding, lubricating, colouring, and/or flavouring agents as well as absorbents, absorption enhancers, humefactants, preservatives and the like, and/or any components normally found in corresponding products. The pharmaceutical compositions may be produced by any conventional processes known in the art.

Compositions may be produced by processes well known in the art, e.g. by means of conventional mixing, dissolving, encapsulating, entrapping, lyophilizing, emulsifying and granulating processes. The proper formulation is dependent upon the route of administration chosen, and the pharmaceutical composition can be formulated for immediate release or slow release (e.g. in order to prolong the therapeutic effect and/or improve tolerability). The pharmaceutical composition may be in any form, such as in a solid, semisolid or liquid form, suitable for administration. A formulation can be selected from a group consisting of, but not limited to, solutions, emulsions, suspensions, tablets, pellets, sprays, suppositories and capsules.

Amounts and regimens for therapeutic administration of the compound having formula (I) can be determined readily by those skilled in the clinical art of treating microbial infections. Generally, the dosage of the compound varies depending on multiple factors such as age, gender, other possible treatments, infection in question and severity of the symptoms. For administration of the compound of the present invention a typical dose may be in the range of 0.5 to 2000 mg/kg, more specifically in the range of 5 to 200 mg/kg. A desired dosage can be administered in one or more doses at suitable intervals to obtain the desired results. In a specific embodiment of the invention the composition is administered once or several times. Only one administration may have therapeutic effects, but specific embodiments of the invention require several administrations during the treatment period. The length of the treatment period may vary, and may, for example, last from a single administration to 1-24 months, one to five years or even more.

In a specific embodiment of the invention a molar concentration of the compound of formula (I) of the invention is about 0.5-1000 μM or about 0.5-400 μM. In another specific embodiment of the invention a molar concentration of the compound of formula (I) of the invention is about 0.5-200 μM, about 5-150 μM, about 7-130 μM, about 25-135 μM or about 9-65 μM.

In one embodiment of the invention a subject to be treated or prevented with the compound of the invention having formula (I) is a human or an animal in need of a treatment or prevention. Most preferably a subject is a human patient suffering from bacterial biofilms colonization or other microbial infections. Also any animal, such as a pet, domestic animal or production animal may be a subject of the present invention. The term “subject” includes organisms capable of suffering from bacterial infections.

Before classifying a subject as suitable for the therapy of the present invention, the clinician may for example study any symptoms or assay any disease markers of the subject. Based on the results deviating from the normal, the clinician may suggest the compound having formula (I) of the present invention for treatment.

Any conventional method may be used for administration of the compound or a pharmaceutical composition to a subject. The route of administration depends on the formulation or form of the composition, the disease, the patient, and other factors, and the route of administration can be selected from the group consisting of intra-arterial, intravenous, intracavitary, intracranial, intramuscular, subcutaneous, intramedullary, intrathecal, intraventricular, intranasal, intraocular or intraperitoneal injection, or an oral, rectal, intravaginal, transmucosal, transdermal, suppository, inhalable or topical administration.

Additionally, the administration of the compound can be combined to the administration of other therapeutic agents. The administration can be simultaneous, separate or sequential. In a specific embodiment of the invention the composition is administered before, after or concurrently with another antimicrobial agent. The administration may also be combined to other forms of therapy, such as surgery. Antibacterial or antimicrobial agents suitable for use in combination with compounds of the present invention include e.g. fusidic acid, rifampicin, vancomycin, teicoplanin, cephalosporin, lincosamide (e.g. clindamycin or lincomycin), cotrimoxazole, linezolid, and/or quinupristin/dalfopristin. A person skilled in the art of treating infections may easily recognize additional, clinically relevant agents that may be useful.

Any method or use of the invention may be performed either in vivo, ex vivo or in vitro.

### Non-pharmaceutical Methods and Uses

Microbes (i.e. micro-organisms including bacteria) occur everywhere. However, the amount of microbes, specifically pathogenic microbes, should be reduced in certain situations. For example water or food contaminated with too many or disease-causing microbes may cause an epidemic. Also, in hospitals (specifically operating rooms) and laboratories microbial infections or contaminations should be avoided. Destroying microbes is not an easy task because many microbes and especially biofilms have exceptional resilience to removal by disinfectants and mechanical cleaning processes. Indeed, more effective antimicrobial agents are needed on market.

The present invention provides an effective application for preventing microbes on surfaces. A method of coating a surface of a material comprises applying the compound of formula (I) or a composition comprising the compound of formula (I) to the surface of the material. As used herein “surface” refers to either the outer or inner surface. Also the composition comprising the compound of formula (I) may be applied into a material. As used herein “material” refers to any substance, product, device or medicament comprising a solid surface suitable for coating or having structure suitable for including the composition of the present invention. Most specifically the material to be coated or the material wherein the composition may be applied can be selected from the group consisting of medical devices such as catheters, prostheses, heart replacement valves, implants, contact lenses and surgical sutures, water filtration systems, ship hulls, textiles, furniture, food and food-related related surfaces, pharmaceuticals and devices for drug delivery, dressings, coatings, anti-biofilm agents, laboratory devices, biosensors, anti-biofilm agents for laboratory use, materials for patterned cell culture, diagnostic kits, cleaning solutions or desinfectants.

As used herein “a coating” refers to any composition forming or suitable for forming a coating on the surface of material. According to the present invention the coating comprises a compound of formula (I).

According to the present invention one, two or several compounds of the present invention (either having same or different formulas) may be included in a non-pharmaceutical composition. The composition suitable for coating or to be added into the material comprises at least one compound of the invention, or salt forms or selected combinations thereof. In addition a composition may also comprise any other agents, such as at least one selected from the group consisting of a solvent, diluent, carrier, buffer, excipient, adjuvant, antiseptic, and a filling, stabilizing, thickening, wetting, dispersing, solubilizing, suspending, emulsifying, binding, disintegrating, encapsulating, coating, embedding, lubricating, colouring, and flavouring agent as well as an absorbent, absorption enhancer, humectant, preservative and the like, and any components normally found in corresponding coating products. The non-pharmaceutical compositions may be produced by any conventional processes known in the art.

Compositions may be produced by processes well known in the art, e.g. by means of conventional mixing, dissolving, encapsulating, entrapping, lyophilizing, emulsifying and granulating processes. The proper formulation is dependent upon the application or coating method chosen. The composition may be in any form, such as in a solid, semisolid or liquid form, suitable for coating. A formulation can be selected from a group consisting of, but not limited to, powders, solutions, emulsions, colloidal suspensions, tablets, pellets, aerosols, capsules, and gels.

Amounts and regimens for applying the composition or compound having formula (I) on the surface of a material or within the material can be determined readily by those skilled in the art. Generally, the amount and form of the composition varies depending on multiple factors such as the type and material of the surface to be coated or the material to be applied with the composition. A composition can be applied during one or more application times at suitable intervals to obtain the desired result. In a specific embodiment of the invention the composition is applied once or several times. The length of the suitable interval may vary, and may, for example, last from few minutes to several days or weeks.

Methods suitable for applying a composition of the present invention to the surface of the material include but are not limited to dipping, printing, spraying, painting and grafting onto/from (including the use of chemical or bio-chemical spacers). Conventional coating methods are well known to a person skilled in the art. Methods suitable for applying a composition of the present invention into a material include but are not limited to mixing, printing, injecting, absorbing and moulding. Conventional application methods are well known to a person skilled in the art.

In a specific embodiment of the invention a molar concentration of the compound of formula (I) of the invention is about 0.5-1000 μM or about 0.5-400 μM. In another specific embodiment of the invention a molar concentration of the compound of formula (I) of the invention is about 0.5-200 μM, about 5-150 μM, about 7-130 μM, about 25-135 μM or about 9-65 μM.

Additionally, the application of the compound of the present invention can be combined to the application of other agents such as antimicrobial agents or coating agents. The administration can be simultaneous, separate or sequential. In a specific embodiment of the invention the composition is applied before, after or concurrently with another antimicrobial agent or coating agent. Antibacterial or antimicrobial agents suitable for use in combination with compounds of the present invention include e.g. fusidic acid, rifampicin, vancomycin, teicoplanin, cephalosporin, lincosamide (e.g. clindamycin or lincomycin), cotrimoxazole, linezolid, and/or quinupristin/dalfopristin. A person skilled in the art of antimicrobial agents may easily recognize additional, relevant agents that may be useful.

It will be obvious to a person skilled in the art that, as the technology advances, the inventive concept can be implemented in various ways. The invention and its embodiments are not limited to the examples described above but may vary within the scope of the claims.

### EXAMPLES

The abietane-type diterpenes reported herein refer to the general formula (I) and examples of their synthesis below. Details of the synthetic procedures are provided at the end of the Examples chapter. Used reagents and conditions: i. 1-Ethyl-3-(3-dimethylaminopropyl)carbodiimide hydrochloride (EDC.HCl), Hydroxybenzotriazole (HOBt), N,N-Diisopropylethylamine (DIPEA), Amino acid alkyl ester or di-/tripeptide alkyl ester, dimethylformamide (DMF), r.t.; ii. NaOH (4 M), THF:MeOH, 0° C. to r.t.; iii. EDC.HCl, HOBt, NH₃ (aq), DMF, r.t. iv. CrCO₃, ethyl acetate, acetic acid, 50° C.; v. NH₂OH.xHCl, pyridine, EtOH, 100° C.

Overall, our synthetic strategy involved the coupling of several aminoacid or peptidic residues to the core of dehydroabietic acid by means of easy and relatively inexpensive carbodiimide coupling reactions, in high yields. Both the starting materials and the aminoacid and peptidic building blocks are commercially available at affordable prices. Further chemical modifications included the deprotection of the alkyl side chains of the initially prepared derivatives by alkaline hydrolysis, oxime synthesis from the corresponding carbonyl precursors, and oxidations with m-chloroperoxybenzoic acid to afford the N-oxide derivatives. Our synthetic strategy can in addition easily accommodate the classical chemical modifications at other positions of the dehydroabietic acid core for instance, the introduction of hydroxyl, ester, aldehyde or amine functions at positions 15 or 12.

Anti-biofilm profiling of the abietane-type diterpenoids using a phenotypic assay that measures cellular viability of S. aureus ATCC 25923 biofilms with a redox dye as reported by Sandberg et al. 2009, showed that several compounds displayed significant activity at 400 μM, as depicted on Table 1.

TABLE 1
Anti-biofilm screening data at 400 μM) against S. aureus ATCC 25923.

% Inhibition 400 μM

| Compound | PRE | POST |
|---|---|---|
| 18 | 100.0 | 99.2 |
| 19 | 99.8 | 98.9 |
| 20 | 100.1 | 99.8 |
| 21 | 100.1 | 99.6 |
| 22 | 100.1 | 99.6 |
| 23 | 101.8 | 92.6 |
| 24 | 101.8 | 26.3 |
| 25 | 102.0 | 101.6 |
| 31 | 102.3 | 102.0 |
| 32 | 101.9 | 102.5 |
| 34 | 101.9 | 87.7 |

Reconfirmation studies showed that at least for six of them the activity was maintained at 100 μM. We selected six of the active compounds in the post-exposure assays namely, compounds 20, 21, 22, 25, 31 and 32 for the follow-up studies. Compounds 20, 21, 22, 25, 31 and 32 were able to prevent bacterial colonization with potencies on the micromolar range, and the most active was 22 (IC₅₀=4.2 μg/mL), as depicted on Table 2. In this mode of the assay, the interaction of the compounds occurs initially with single-cell bacteria (that is, a planktonic solution) before biofilms are formed. Therefore, we tested additionally if the compounds could exert antibacterial activity against single-cell bacteria, in the absence of biofilms. Based solely on conventional MIC and MBC values, antibacterial activities against single-cell bacteria were also high for the derivatives. MIC values for compound 20, 21, 25, 31 and 32 were: 33.1 μg/mL; 22.4 μg/mL, 29.3 μg/mL, 31.3 μg/mL, and 46.8 μg/mL which are all under 100 μg/mL. The MIC value for the very potent compound 22 was found to be 6.8 μg/mL, thus lower than 10 μg/mL, as desirable of isolated compounds according to Rios and Recio (2005). The antibacterial activity order 22>21>25>31>20>32 correlated very well with the measured activity in preventing biofilm formation. Thus, it is plausible to assume that these compounds inhibit biofilm formation by killing single-cell bacteria before it reaches the substrate to initiate the biofilm formation process. In particular, we showed that compound 22 is a highly potent inhibitor of biofilm colonization that can also act against planktonic bacteria.

TABLE 2
Anti-biofilm potencies of the most active derivatives against S. aureus
ATCC 25923 strain. All results are expressed in μM and between
parentheses in μg/mL. For anti-biofilm activities, concentrations causing
50% of inhibition of biofilm viability are shown, measured prior to and
after biofilms have been formed. The post-exposure effect was measured
24 hours after adding the compounds to the existing biofilms.

| | Anti-biofilm activity, expressed as μM (μg/mL) | |
|---|---|---|
| | Potency, IC₅₀ | Potency, IC₅₀ |
| Compound | Prior-to-biofilm formation | Post-biofilm formation |
| 20 | 62.2 (25.7) | 121.3 (50.2) |
| 21 | 35.5 (15.9) | 108.7 (48.7) |
| 22 | 9.4 (4.2) | 27.9 (12.7) |
| 25 | 33.2 (16.2) | 86.1 (41.9) |
| 31 | 37.4 (16.7) | 98.2 (44.0) |
| 32 | 60.6 (28.3) | 145.3 (67.9) |
| Penicillin G | 0.13 (0.048) | 57% inhibition at 400 μM |
| Vancomycin | 0.71 (1.03) | 25% inhibition at 400 μM |

Most importantly, from a translational perspective, these compounds were also found to significantly reduce the viability of established S. aureus biofilms (22>25>31>21>20>32). Thus, their activity was maintained even in the presence of chemotolerant S. aureus biofilms (notice that penicillin G and vancomycin can efficiently prevent biofilm formation, but they cannot significantly decrease the viability of existing biofilms more than 60% even if added at high micromolar concentrations). In fact, only 2-3 fold-higher concentrations of the compounds were needed to kill existing biofilms, in comparison to inhibiting bacterial colonization. The lower chemotolerance of in vitro S. aureus biofilms to these abietane-type diterpenoids is an advantageous factor that significantly benefits their future applications. Anti-biofilm potencies were all in the micromolar range and they were particularly significant for compound 22. This activity in existing biofilms is comparable to the most active anti-biofilms compounds reported in the literature, so far. In particular, they are now, to the best of our knowledge, the most active anti-biofilm abietane-type diterpenoids (see section 10 for references). The activity was also confirmed against S. aureus Newman strain. Potency values calculated for the S. aureus Newman (Table 3) were very similar to the values registered for S. aureus ATCC 25923.

TABLE 3
Anti-biofilm potencies of the most active derivatives against S. aureus
Newman. All results are expressed in μM and between parentheses
in μg/mL. Concentrations causing 50% of inhibition of biofilm viability
are shown, measured prior to and after biofilms have been formed.

| | Anti-biofilm activity, expressed as μM (μg/mL) | |
|---|---|---|
| | Potency, IC₅₀ | Potency, IC₅₀ |
| Compound | Prior-to-biofilm formation | Post-biofilm formation |
| 20 | 51.8 (21.4) | 134.5 (55.6) |
| 21 | 35.9 (16.1) | 83.7 (37.5) |
| 22 | 7.9 (3.6) | 48.2 (21.9) |
| 25 | 20.9 (10.2) | 71.7 (34.9) |
| 31 | 36.2 (16.2) | 110.3 (49.4) |
| 32 | 63.5 (29.7) | 93.1 (43.5) |
| Penicillin G | 0.27 (0.090) | 73% inhibition at 400 μM |
| Vancomycin | 1.3 (1.88) | 37.9% inhibition at 400 μM |

Four of the compounds were also tested against gram-negative E. coli biofilms as well as other Staphylococcal strains, at the corresponding IC₅₀ values measured against S. aureus ATCC 25923 (Table 4). The tested compounds were less active against E. coli (lower than 50% inhibition in all cases), indicating that most likely these compounds are selective towards gram-positive bacteria. Nonetheless, all the compounds presented below were found active against S. epidermidis (ATCC 12228 and ATCC 35984) strains (Table 3). Altogether, these results demonstrate that the compounds do exhibit a wider spectrum of anti-biofilm effects against Staphylococcus spp.

TABLE 4
Anti-biofilm activity of four selected compounds against a panel of
representative strains.

| | | |
|---|---|---|
| | Inhibition | |
| Compound | percentage (±SD) | Inhibition percentage (±SD) |
| (IC₅₀ conc.) | Prior-to-biofilm formation | Post-biofilm formation |

Anti-biofilm activity against E. coli XL1 blue

| | | |
|---|---|---|
| 20 | 14.2 (±4.5) | 39.9 (±4.6) |
| 21 | 7.4 (±2.7) | 30.4 (±6.1) |
| 22 | 5.2 (±11.3) | 14.2 (±13.1) |
| 25 | 8.7 (±5.6) | 6.7 (±7.2) |

Anti-biofilm activity against S. epidermidis
ATCC 12228

| | | |
|---|---|---|
| 20 | 62.4 (±2.2) | 12.9 (±5.1) |
| 21 | 57.3 (±4.6) | 46.9 (±9.9) |
| 22 | 42.3 (±9.5) | 4.4 (±5.8) |
| 25 | 70.3 (±5.6) | 48.8 (±10.3) |

Anti-biofilm activity against S. epidermidis
ATCC 35984

| | | |
|---|---|---|
| 20 | 53.4 (±4.9) | −12 (±6.0) |
| 21 | 58.1 (±4.1) | 44.4 (±8.4) |
| 22 | 48.8 (±4.8) | 1.9 (±5.7) |
| 25 | 66.9 (±0.9) | 38.0 (±9.3) |

The viability of S. aureus (ATCC 25923) biofilms left upon exposure to compounds 20, 21, 22 and 25 was determined using viable plate counts and calculating the Log Reduction (Log R) value. This procedure involves scrapping the biofilms off the substrate, disaggregating them by sonication and plating the resulting suspension in agar. The method is highly laborious but it is the gold standard for the quantification of anti-biofilm efficacy (Pitts et al. 2003). The four compounds caused Log Reduction values ranging from 2.3 to 6.2, when tested at 400 μM (Table 5). Of note, a log R value of 2 represents a reduction of 99% of the viable cells in the biofilms, and typically, a log R of 3 is considered a relevant indicator of the compound efficacy as an anti-biofilm agent.

TABLE 5
Anti-biofilm efficacy of four of the most active derivatives measured
against S. aureus ATCC 25923 strain. The assay uses viable plate
counts and calculation of the Log Reduction (Log R) value.

| Compound | Anti-biofilm activity in S. aureus ATCC 25923 |
|---|---|
| (400 μM) | Log Reduction (average ±SD) |
| 20 | 3.9 (±0.4) |
| 21 | 4.3 (±0.2) |
| 22 | 2.3 (±0.1) |
| 25 | 6.2 (±0.4) |
| Penicillin G | 1.0 (±0.1) |

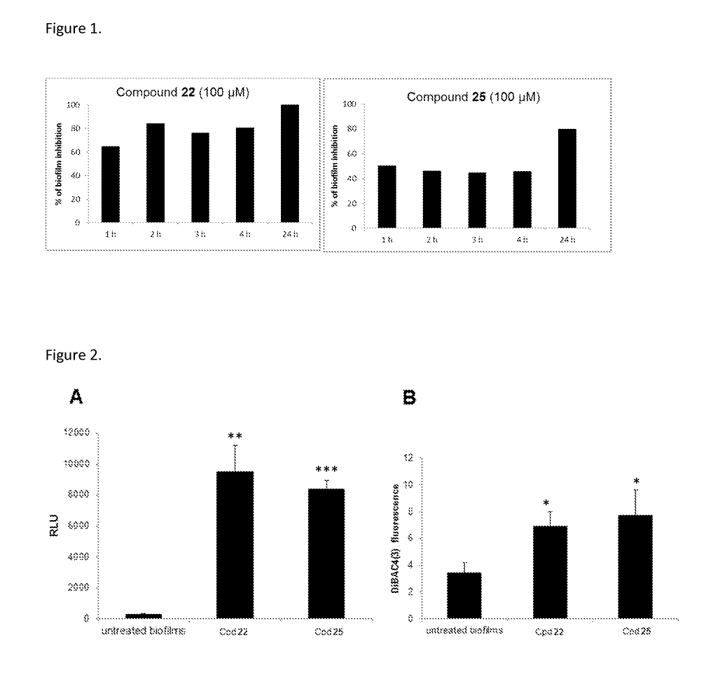

Based overall on all the results presented earlier (including both potency and efficacy studies), compounds 22 and 25 were selected for further studies. The action of all compounds on the biofilms seems to occur fast. Within the first hour a reduction of nearly 50% of the viable biofilm cells was detected (FIG. 1). In both cases, after 24 hours, inhibition of over 80% of viable biofilm cells was observed, in agreement with previous studies (IC₅₀ values are both under 100 μM for post-exposure conditions). This rapid biofilm killing kinetics may be indicative of a membrane-targeting mechanism.

ATP release from the biofilms after 1 h exposure to compounds 22 and 25 at 100 μM was quantified on the culture media, using the BacTiter-Glo bioluminescent assay. It was confirmed that both compounds cause a highly significant ATP leakage from S. aureus biofilms, which would thus explain the fast killing kinetics (FIG. 2A). ATP release can be often associated to membrane depolarization events. Here, such events were followed by the increase in fluorescence emission of the DiBAC4(3) probe, as in Okuda et al. (2013). Both compounds caused membrane depolarization, upon 1 h exposure to the biofilms (as earlier, FIG. 2B).

Tolerability of mammalian cells to the four most active compounds was also studied using HL cells (originating from human respiratory tract). Viability values were measured in acute conditions (24 hours exposure) using the resazurin assay as in Karlsson et al, 2012 (Eur J Pharm Sci. 2012 Aug 30;47(1):190-205). Concentrations of up to 100 μM of compounds 20 and 21 did not cause statistically significant cytotoxicity. The least tolerated molecule was 25, as only 23% (±5.9) of cells remained viable upon exposure to 100 μM. However, concentrations up to 30 μM caused no cytotoxicity (90.2% of viable cells, ±0.5).

Additionally, the biocompatibility index (BI) was calculated for compound 22, to assess the overall impact of its antimicrobial activity, using an adaptation of the equation described for antiseptics by Muller and Kramer, 2008. BI, as originally defined by these authors, is a dimensionless parameter resulting from the ratio of the in vitro cytotoxicity values (in this case, half-lethal concentrations, LC₅₀) to the concentration of the compound causing a 3-log reduction in the viable counts of suspended bacteria. Compounds with BI<1 are deemed as less promising, due to their potential toxic effects. Thus BI is a very useful tool for the quick exclusion of undesired toxic scaffolds or weak hits in early stage of development of anti-biofilm molecules. For the determination of the BI, acute cytotoxicity of compound 22 was measured in the same cell line described earlier (HL cells), using the resazurin assay as in Karlsson et al, 2012. The ratio of the half lethal concentration (LC₅₀) and the concentration causing 3-log reduction in the planktonic bacterial burden was calculated (both expressed in ma/L), as below:

$$BI = \frac{45.2 mg \text{/} L}{22.3 mg \text{/} L} = 2.1 .$$

The BI value of compound 22 was clearly higher than 1, which is indicative of an adequate combination of an effective anti-biofilm activity with a low cytotoxicity. It is thus expected that toxic effects can be minimized in the host organisms exposed to compound 22.

We have therefore been able to identify the L-amino acids leucine (compound 20) and phenylalanine (compound 21), the unusual β-cyclohexyl-L-alanine (compound 22 and 32) and the D-amino acid tryptophane (compound 25) and phenylalanine (compound 31) as examples of suitable side-chains for the parent dehydroabietic acid (1), that resulted in the production of potent anti-biofilm compounds. The most potent derivative 22 bears the unusual β-cyclohexyl-L-alanine amino acid as side chain with a free carboxyl group. In fact, among the compounds tested, we found that a free carboxyl group on the amino acid residue was crucial for the bioactivity. In addition, the absence of aromaticity in the side chain of 22 resulted in much higher potency when compared to the aromatic derivative 21. Of note, our design strategy proved effective not only using the natural amino acids as side chains but also with D-amino acids (which typically retain the full activity of their L-counterparts) as well as with unusual amino acids. Both D- and unusual amino acids have the competitive advantage of resistance to enzymatic proteolysis. Our mechanistic studies point out to the bacterial membrane as one (likely primary) target of the anti-biofilm effects of these derivatives, in a manner resembling the action mechanisms of AMPs. However, while biomedical applications of AMPs are typically hampered by their large sizes, susceptibility to enzymatic degradation, and higher production costs, these are all limitations that can be overcome by these abietane-type diterpenoids.

### Indications

The compounds described in this invention display improved anti-biofilm effects when compared to the parent compound 1. The simple but innovative chemical strategy consisting of combining two active scaffolds from natural sources resulted in a significant enhancement of the activity against S. aureus biofilms of the final compounds reported herein. These exhibit potencies that are remarkably high when compared to the available repertoire of compounds active against bacterial biofilms. Their synthesis relies on the use of abundant natural products and is facile, inexpensive, and high-yielding.

These compounds can be regarded as potent molecular probes for further biofilm studies or as lead structures for the development of new anti-biofilm agents with applicability in many health and industrial settings. Resin acid-containing preparations are currently being commercialized for use as human health products for wound-healing and treatment of nail fungal infections (Repolar Oy), thus highlighting the feasibility of the straightforward commercial applications for the compounds described in this patent application. The fact that the parent resin acids are naturally occurring and highly abundant from the rosin of coniferous trees, the most abundant trees in Finnish forests, highlights the value added by our work to the Finnish bioeconomy in particular.

### Coating

One, two or several compounds of formula (I) (either having same or different formulas) (e.g. compounds 20, 21, 22 or 25 or any combination thereof) are included in a non-pharmaceutical composition. In addition a composition comprises any other agents, such as at least one selected from the group consisting of a solvent, diluent, carrier, buffer, excipient, adjuvant, anti-septic, and a filling, stabilizing, thickening, wetting, dispersing, solubilizing, suspending, emulsifying, binding, disintegrating, encapsulating, coating, embedding, lubricating, colouring, and flavouring agent as well as an absorbent, absorption enhancer, humectant, preservative and the like, and any components normally found in corresponding coating products. The non-pharmaceutical compositions are produced by any conventional processes known in the art e.g. by means of conventional mixing, dissolving, encapsulating, entrapping, lyophilizing, emulsifying and granulating processes.

A composition is applied on the surface of the material or into the material by dipping, printing, spraying, painting, grafting onto/from (including the use of chemical or biochemical spacers), mixing, injecting, absorbing or moulding. Either one or more application times at suitable intervals are utilized to obtain the desired result.

### Example Experimental Data for Some of the Compounds Described Herein

All reagents were obtained from Sigma Aldrich Co or TCI Europe. Dehydroabietic acid (1, 90% purity) was obtained from GmBH. (-)-2-Amino-butyric acid methyl ester hydrochloride, H-Gly-Gly-OEt.HCl, H-Ala-Ala-Ala-OMe acetate salt were obtained from Bachem. β-Cyclohexyl-L-alanine methyl ester hydrochloride was obtained from Novabiochem. For thin layer chromatography (TLC) analysis, Kieselgel 60 HF254/Kieselgel 60G was used (Merk). Flash Column Chromatography (FCC) was made with a Biotage High-Performance Flash Chromatography Sp4-system (Uppsala, Sweden) using a 0.1-mm pathlength flow cell UV-detector/recorder module (fixed wavelength: 254 nm), and 12-mm or 25-mm flash cartridges. Melting points were recorded with an Electrothermal capillary tube melting point apparatus and are uncorrected. IR spectra were obtained using a Vertex 70 (Bruker Optics Inc., MA, USA) FTIR instrument. The FTIR measurements were made directly in solids with a horizontal Attenuated Total Reflectance (ATR) accessory (MIRacle, Pike Technology, Inc, WI, USA). The transmittance spectra were recorded at a 4 cm⁻¹ resolution between 4000 and 600 cm⁻¹ using the OPUS 5.5 software (Bruker Optics Inc., MA, USA). NMR spectra were obtained using a Varian Mercury Plus 300 spectrometer, in CDCl₃ or DMSO-d₆, with tetramethylsilane (TMS) as the internal standard. The chemical shifts were reported in parts per million (ppm) and on the δ scale from TMS as an internal standard. The coupling constants J are quoted in hertz (Hz). ESI-MS was performed by direct injection using a Synapt G2 HDMS (Waters, MA, USA) instrument.

Methyl N-(abiet-8,11,13-trien-18-oyl) Glycinate (3)

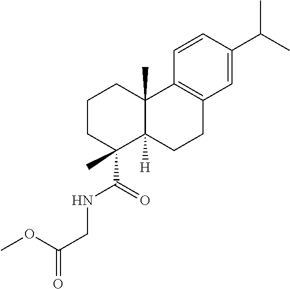 [SMILES: COC(=O)CNC(=O)[C@]1(C)CCC[C@]2(C)c3ccc(C(C)C)cc3CC[C@@H]12]

Compound 1 (1 g; 3.33 mmol) was dissolved in DMF (10 mL), at room temperature. EDC hydrochloride (956 mg; 5 mmol) and HOBt monohydrate (676 mg; 5 mmol) were added and the mixture was left to agitate for 1 hour. Glycine methyl ester hydrochloride (628 mg; 5 mmol) and DIPEA (1.76 mL; 10 mmol) were then added and the mixture was left to agitate for another hour after which the reaction was complete. The reaction was suspended by addition of diethyl ether (200 mL) and water (40 mL). The aqueous phase was further extracted with diethyl ether (2×100 mL). The resulting organic phase was washed with aqueous HCl (50 mL), saturated NaHCO₃ solution (50 mL), water (50 mL), and brine (50 mL), dried with Na₂SO₄, filtered, and evaporated to dryness. Compound 3: (1.2 g, 97%). Mp 46-48° C. IR (ATR) 3376, 1758, 1640, 1519, 1205, 1179, 820 cm⁻¹. ¹H-NMR (300 MHz, CDCl₃) δ 1.21 (s, 3 H), 1.23 (s, 6 H), 1.32 (s, 3 H), 2.14 (dd, J₁=12.4 Hz and J₂=2.1 Hz, 1 H), 2.31 (d, J=12.9 Hz, 1 H), 2.86 (m, 3 H), 3.12 (s, 3 H, OCH₃), 4.04 (d, J=5.1 Hz, 2 H, −NHCH₂), 6.32 (brs, 1 H, NH), 6.86 (s, 1 H, 14-H), 6.98 (d, J=8.1 Hz, 1 H, aromatic-H), 7.16 (d, J=8.1 Hz, 1 H, aromatic-H). ¹³C-NMR (75 MHz, CDCl₃) δ 16.6, 18.9, 21.3, 24.1, 25.4, 30.1, 33.6, 37.3, 37.4, 38.1, 41.7, 45.7, 47.5, 52.5, 124 (aromatic-C), 124.2 (aromatic-C), 127.1 (aromatic-C), 134.8 (aromatic-C), 145.9 (aromatic-C), 147.1 (aromatic-C), 170.9 and 178.9 (COOCH₃ and CONH). HRMS m/z: calcd. for C₂₃H₃₄NO₃ 372.2539 [M+1]⁺, found 372.2538.

Methyl N-(abiet-8,11,13-trien-18-oyl) L-valinate (4)

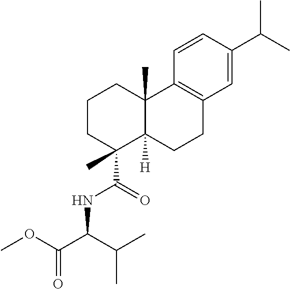 [SMILES: COC(=O)[C@@H](NC(=O)[C@]1(C)CCC[C@]2(C)c3ccc(C(C)C)cc3CC[C@@H]12)C(C)C]

Following the procedure for compound 3, compound 4 was prepared from 1 (250 mg; 0.83 mmol), EDC hydrochloride (239 mg; 1.25 mmol), HOBt monohydrate (169 mg; 1.25 mmol), valine methyl ester hydrochloride (209 mg; 1.25 mmol), and DIPEA (0.44 mL; 2.5 mmol), in DMF (2.5 mL). Compound 4: (309 mg, 90%). Mp 106-107° C. IR (ATR) 3454, 1737, 1658, 1498, 1305, 821 cm⁻¹. ¹H-NMR (300 MHz, CDCl₃) δ 0.93 (dd, J₁=10.8 Hz and J₂=6.9 Hz, 6 H, —CH(CH₃)₂), 1.21 (s, 3 H), 1.23 (s, 3 H), 1.24 (s, 3 H), 1.31 (s, 3 H), 2.11 (dd, J₁=12.4 Hz and J₂=2 Hz, 1 H), 2.18 (m, 1 H), 2.33 (d, J=13 Hz, 1 H), 2.88 (m, 3 H), 3.74 (s, 3 H, OCH₃), 4.58 (dd, J₁=8.4 Hz and J₂=4.8 Hz, 1 H, —NHCH—), 6.23 (d, J=8.4 Hz, 1 H, NH), 6.89 (s, 1 H, 14-H), 7.0 (dd, J₁=8.1 Hz and J₂=1.8 Hz, 1 H, aromatic-H), 7.17 (d, J₁=8.1 Hz, 1 H, aromatic-H). ¹³C-NMR (75 MHz, CDCl₃) δ 16.4, 17.9, 18.7, 19, 21.2, 23.9, 23.9, 25.3, 30, 31.2, 33.4, 37.2, 37.5, 38, 45.7, 47.4, 52, 57, 123.8 (aromatic-C), 124.1 (aromatic-C), 126.9 (aromatic-C), 134.6 (aromatic-C), 145.7 (aromatic-C), 146.8 (aromatic-C), 172.8 and 178.2 (COOCH₃ and CONH). HRMS m/z: calcd. for C₂₆H₄₀1\10₃ 414.3008 [M+1]⁺, found 414.3009.

Methyl N-(abiet-8,11,13-trien-18-oyl) L-ethylglycinate (5)

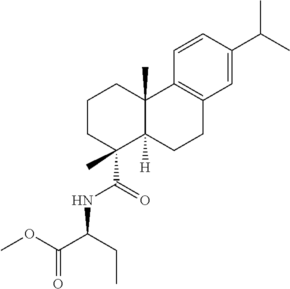 [SMILES: CC[C@H](NC(=O)[C@]1(C)CCC[C@]2(C)c3ccc(C(C)C)cc3CC[C@@H]12)C(=O)OC]

Following the procedure for compound 3, compound 5 was prepared from 1 (250 mg; 0.83 mmol), EDC hydrochloride (239 mg; 1.25 mmol), HOBt monohydrate (169 mg; 1.25 mmol), (-)-2-aminobutyric acid methyl ester hydrochloride (276 mg; 1.8 mmol), and DIPEA (0.44 mL; 2.5 mmol), in DMF (2.5 mL). Compound 5: (307 mg, 92%). Mp 51-53° C. IR (ATR) 3361, 1743, 1637, 1517, 1207, 821 cm⁻¹. ¹H-NMR (300 MHz, CDCl₃) δ 0.92 (t, J=7.5 Hz, 3 H, —CH₂CH₃), 1.21 (s, 3 H), 1.23 (s, 3 H), 1.24 (s, 3 H), 1.31 (s, 3 H), 2.11 (dd, J₁=12.4 Hz and J₂=1.8 Hz, 1 H), 2.32 (d, J=12.9 Hz, 1 H), 2.86 (m, 3 H), 3.74 (s, 3 H, OCH₃), 4.58 (m, 1 H, —NHCH—), 6.25 (d, J=7.5 Hz, 1 H, NH), 6.88 (s, 1 H, 14-H), 7.0 (d, J=8.1 Hz, 1 H, aromatic-H), 7.17 (d, J=8.1 Hz, 1 H, aromatic-H). ¹³C-NMR (75 MHz, CDCl₃) δ 9.6, 16.3, 18.7, 21.1, 23.9, 23.9, 25.2, 25.5, 30, 33.4, 37.1, 37.4, 38, 45.6, 47.3, 52.1, 53.3, 123.8 (aromatic-C), 124 (aromatic-C), 126.9 (aromatic-C), 134.6 (aromatic-C), 145.7 (aromatic-C), 146.8 (aromatic-C), 173.1 and 178 (COOCH₃ and CONH). HRMS m/z: calcd. for C₂₅H₃₈NO₃ 400.2852 [M+1]⁺, found 400.2853.

Methyl N-(abiet-8,11,13-trien-18-oyl) L-leucinate (6)

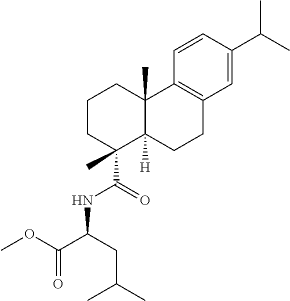 [SMILES: COC(=O)[C@H](CC(C)C)NC(=O)[C@]1(C)CCC[C@]2(C)c3ccc(C(C)C)cc3CC[C@@H]12]

Following the procedure for compound 3, compound 6 was prepared from 1 (250 mg; 0.83 mmol), EDC hydrochloride (239 mg; 1.25 mmol), HOBt monohydrate (169 mg; 1.25 mmol), leucine methyl ester hydrochloride (226 mg; 1.24 mmol), and DIPEA (0.44 mL; 2.5 mmol), in DMF (2.5 mL). Compound 6: (305 mg, 86%). Mp 117-118° C. IR (ATR) 3346, 1755, 1629, 1527, 1155, 821 cm⁻¹. ¹H-NMR (300 MHz, CDCl₃) δ 0.95 (d, J=5 Hz, 6 H, —CH₂CH(CH₃)₂), 1.21 (s, 3 H), 1.23 (s, 3 H), 1.24 (s, 3 H), 1.30 (s, 3 H), 2.07 (dd, J₁=12.4 Hz and J₂=2 Hz, 1 H), 2.33 (d, J=13.3 Hz, 1 H), 2.87 (m, 3 H), 3.73 (s, 3 H, OCH₃), 4.63 (m, 1 H, —NHCH—), 6.07 (d, J=8 Hz, 1 H, NH), 6.89 (s, 1 H, 14-H), 7.0 (dd, J₁=8.2 Hz and J₂=1.8 Hz, 1 H, aromatic-H), 7.17 (d, J=8.2 Hz, 1 H, aromatic-H). ¹³C-NMR (75 MHz, CDCl₃) δ 16.3, 18.7, 21.1, 21.9, 22.8, 23.9, 23.9, 25, 25.2, 29.9, 33.4, 37.1, 37.4, 38, 41.5, 45.8, 47.2, 50.8, 52.1, 123.8 (aromatic-C), 124 (aromatic-C), 126.9 (aromatic-C), 134.6 (aromatic-C), 145.7 (aromatic-C), 146.9 (aromatic-C), 173.7 and 178.1 (COOCH₃ and CONH). HRMS m/z: calcd. for C₂₇H₄₂NO₃ 428.3165 [M+1]⁺, found 428.3169.

Methyl N-(abiet-8,11,13-trien-18-oyl) L-phenylalaninate (7)

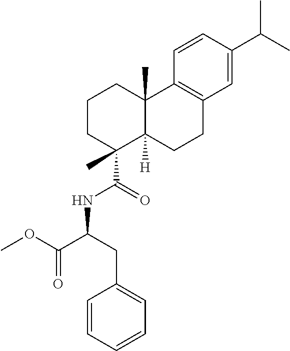 [SMILES: COC(=O)[C@H](Cc1ccccc1)NC(=O)[C@]1(C)CCC[C@]2(C)c3ccc(C(C)C)cc3CC[C@@H]12]

Following the procedure for compound 3, compound 7 was prepared from 1 (250 mg; 0.83 mmol), EDC hydrochloride (239 mg; 1.25 mmol), HOBt monohydrate (169 mg; 1.25 mmol), phenylalanine methyl ester hydrochloride (269 mg; 1.25 mmol), and DIPEA (0.44 mL; 2.5 mmol), in DMF (2.5 mL). Compound 7: (372 mg, 97%). Mp 115-117° C. IR (ATR) 3388, 1743, 1639, 1517, 1515, 1215, 821 cm⁻¹. ¹H-NMR (300 MHz, CDCl₃) δ 1.19 (s, 3 H), 1.20 (s, 6 H), 1.22 (s, 3 H), 2.01 (d, J=12.2 Hz, 1 H), 2.27 (d, J=12.8 Hz, 1 H), 2.78 (m, 3 H), 3.11 (ddd, J₁=20.8 Hz, J₂=14 Hz, and J₃=6.3 Hz, 2 H, —CH₂Ph), 3.71 (s, 3 H, OCH₃), 4.90 (m, 1 H, —NHCH—), 6.12 (d, J=7.6 Hz, 1 H, NH), 6.84 (s, 1 H, aromatic-H), 6.97 (d, J₁=8.2 Hz, 1 H, aromatic-H), 7.12 (m, 3 H, aromatic-H), 7.25 (m, 3 H, aromatic-H). ¹³C-NMR (75 MHz, CDCl₃) δ 16.3, 18.7, 20.9, 23.9, 25.2, 29.9, 33.4, 37, 37.2, 37.9, 38, 45.5, 47.2, 52.2, 53, 123.7 (aromatic-C), 124 (aromatic-C), 126.8 (aromatic-C), 127 (aromatic-C), 128.5 (aromatic-C), 129.1 (aromatic-C), 134.6 (aromatic-C), 136 (aromatic-C), 145.6 (aromatic-C), 146.8 (aromatic-C), 172.3 and 177.9 (COOCH₃ and CONH). HRMS m/z: calcd. for C₃₀H₄₀NO₃ 462.3008 [M+1]⁺, found 462.3004.

Methyl N-(abiet-8,11,13-trien-18-oyl) Cyclohexyl-L-alaninate (8)

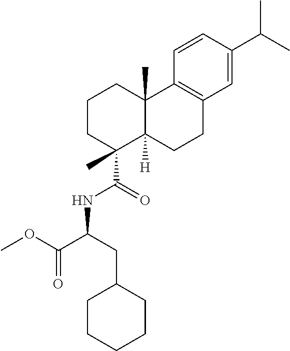 [SMILES: COC(=O)[C@H](CC1CCCCC1)NC(=O)[C@]1(C)CCC[C@]2(C)c3ccc(C(C)C)cc3CC[C@@H]12]

Following the procedure for compound 3, compound 8 was prepared from 1 (250 mg; 0.83 mmol), EDC hydrochloride (239 mg; 1.25 mmol), HOBt monohydrate (169 mg; 1.25 mmol), 13-cyclohexyl-L-alanine methyl ester hydrochloride (277 mg; 1.25 mmol), and DIPEA (0.44 mL; 2.5 mmol), in DMF (2.5 mL). Compound 8: (374 mg, 96%). Mp 106-108° C. IR (ATR) 3344, 1751, 1627, 1525, 1448, 1172, 819 cm⁻¹. ¹H-NMR (300 MHz, CDCl₃) δ 1.21 (s, 3 H), 1.23 (s, 3 H), 1.24 (s, 3 H), 1.30 (s, 3 H), 2.09 (dd, J₁=12.4 Hz and J₂=2.1 Hz, 1 H), 2.32 (d, J=12.9 Hz, 1 H), 2.88 (m, 3 H), 3.72 (s, 3 H, OCH₃), 4.67 (m, 1 H, —NHCH—), 6.07 (d, J=8.2 Hz, 1 H, NH), 6.89 (s, 1 H, 14-H), 7.0 (dd, J₁=8.2 Hz and J₂=1.8 Hz, 1 H, aromatic-H), 7.17 (d, J=8.2 Hz, 1 H, aromatic-H). ¹³0-NMR (75 MHz, CDCl₃) δ 16.3, 18.7, 21.1, 23.9, 23.9, 25.2, 26, 26.2, 26.3, 29.9, 32.3, 33.4, 33.5, 34.4, 37.1, 37.2, 38, 40, 45.7, 47.2, 50.1, 52.1, 123.8 (aromatic-C), 124 (aromatic-C), 126.8 (aromatic-C), 134.6 (aromatic-C), 145.7 (aromatic-)), 146.9 (aromatic-C), 173.9 and 178.1 (COOCH₃ and CONH). HRMS m/z: calcd. for C₃₀H₄₆NO₃ 468.3478 [M+1]⁺, found 468.3477.

Methyl N-(abiet-8,11,13-trien-18-oyl) o-methioninate (9)

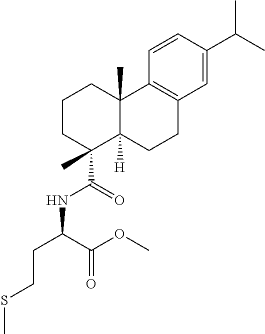 [SMILES: COC(=O)[C@@H](CCSC)NC(=O)[C@]1(C)CCC[C@]2(C)c3ccc(C(C)C)cc3CC[C@@H]12]

Following the procedure for compound 3, compound 9 was prepared from 1 (500 mg; 1.66 mmol), EDC hydrochloride (478 mg; 2.49 mmol), HOBt monohydrate (338 mg; 2.49 mmol), D-methionine methyl ester hydro-chloride (497 mg; 2.49 mmol), and DIPEA (0.88 mL; 5.0 mmol), in DMF (5 mL). Compound 9: (620 mg, 84%). Mp 92-93° C. IR (ATR) 3373, 1737, 1631, 1541, 1228, 821 cm⁻¹. ¹H-NMR (300 MHz, CDCl₃) δ 1.21 (s, 3 H), 1.23 (s, 6 H), 1.31 (s, 3 H), 2.09 (s, 3 H, SCH₃), 2.32 (d, J=12.7 Hz, 1 H), 2.49 (m, 2 H), 2.85 (m, 3 H), 3.76 (s, 3 H, OCH₃), 4.72 (m, 1 H, —NHCH—), 6.49 (d, J=7.5 Hz, 1 H, NH), 6.87 (s, 1 H, 14-H), 6.99 (dd, J₁=8.2 Hz and J₂=1.8 Hz, 1 H, aromatic-H), 7.16 (d, J=8.2 Hz, 1 H, aromatic-H). ¹³C-NMR (75 MHz, CDCl₃) δ 15.6, 16.5, 18.8, 21.26, 24, 25.3, 30.1, 30.2, 31.6, 33.5, 37.2, 37.5, 38.1, 45.8, 47.5, 51.9, 52.5, 124 (aromatic-C), 124.1 (aromatic-C), 127 (aromatic-C), 134.6 (aromatic-C), 145.8 (aromatic-C), 147 (aromatic-C), 172.8 and 178.4 (COOCH₃ and CONH). HRMS m/z: calcd. for C₂₆H₄₀NO₃S 446.2729 [M+1]⁺, found 446.2727.

Methyl N-(abiet-8,11,13-trien-18-oyl) D-tyrosinate (10)

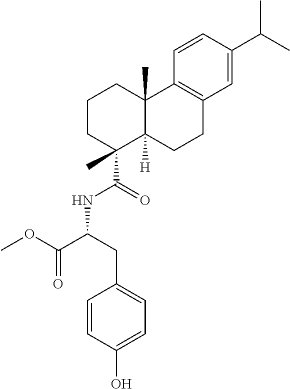 [SMILES: COC(=O)[C@@H](Cc1ccc(O)cc1)NC(=O)[C@]1(C)CCC[C@]2(C)c3ccc(C(C)C)cc3CC[C@@H]12]

Following the procedure for compound 3, compound 10 was prepared from 1 (500 mg; 1.66 mmol), EDC hydrochloride (478 mg; 2.49 mmol), HOBt monohydrate (338 mg; 2.49 mmol), D-tyrosine methyl ester hydrochloride (578 mg; 2.49 mmol), and DIPEA (0.88 mL; 5.0 mmol), in DMF (5 mL). Compound 10: (623 mg, 79%). Mp 83-85° C. IR (ATR) 3354, 1743, 1633, 1514, 1220, 821 cm⁻¹. ¹H-NMR (300 MHz, CDCl₃) δ 1.19 (s, 3 H), 1.20 (s, 3 H), 1.21 (s, 3 H), 1.22 (s, 3 H), 2.07 (dd, J₁=12.4 and J₂=2 Hz, 1 H), 2.28 (d, J=12.9 Hz, 1 H), 2.79 (m, 3 H), 3.01 (m, 2 H), 3.72 (s, 3 H, OCH₃), 4.72 (m, 1 H, —NHCH—), 6.25 (d, J=7.6 Hz, 1 H, NH), 6.71 (m, 2 H, aromatic-H), 6.84 (s, 1 H, aromatic-H), 6.90 (m, 2 H, aromatic-H), 6.97 (dd, J₁=8.2 Hz and J₂=1.7 Hz, 1 H, aromatic-H), 7.13 (d, J=8.2 Hz, 1 H, aromatic-H). ¹³C-NMR (75 MHz, CDCl₃) δ 16.4, 18.8, 21.1, 24.1, 25.3, 30, 33.5, 37.1, 37.2, 38, 45.5, 47.5, 52.4, 53.5, 115.7 (aromatic-C), 123.9 (aromatic-C), 124.1 (aromatic-C), 127 (aromatic-C), 127.2 (aromatic-C), 130 (aromatic-C), 134.7 (aromatic-C), 145.8 (aromatic-C), 146.9 (aromatic-C), 155.6 (aromatic-C), 172.7 and 178.6 (COOCH₃ and CONH). HRMS m/z: calcd. for C₃₀1-1₄₀NC₄ 478.2957 [M+1]⁺, found 478.2954.

Methyl N-(abiet-8,11,13-trien-18-oyl) D-tryptophanate (11)

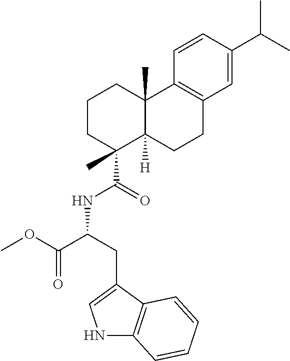 [SMILES: COC(=O)[C@@H](Cc1c[nH]c2ccccc12)NC(=O)[C@]1(C)CCC[C@]2(C)c3ccc(C(C)C)cc3CC[C@@H]12]

Following the procedure for compound 3, compound 11 was prepared from 1 (500 mg; 1.66 mmol), EDC hydrochloride (478 mg; 2.49 mmol), HOBt monohydrate (338 mg; 2.49 mmol), D-tryptophan methyl ester hydrochloride (634 mg; 2.49 mmol), and DIPEA (0.88 mL; 5.0 mmol), in DMF (5 mL). Compound 11: (655 mg, 79%). Mp 80-82° C. IR (ATR) 3315, 1743, 1635, 1498, 1213, 738 cm⁻¹. ¹H-NMR (300 MHz, CDCl₃) δ 1.15 (s, 3 H), 1.18 (s, 3 H), 1.22 (s, 3 H), 1.24 (s, 3 H), 2.06 (m, 1 H), 2.29 (m, 1 H), 2.70 (m, 1 H), 2.83 (m, 1 H), 3.33 (d, J=5.8 Hz, 2 H, —NHCHCH₂—), 3.72 (s, 3 H, OCH₃), 4.96 (m, 1 H, —NHCH—), 6.31 (d, J=7.4 Hz, 1 H, NH), 6.84 (d, J=1.3 Hz, 1 H, aromatic-H), 6.97 (m, 1 H, aromatic-H), 6.97 (m, 1 H, aromatic-H), 7.04 (m, 1 H, aromatic-H), 7.08 (m, 1 H, aromatic-H), 7.17 (m, 2 H, aromatic-H), 7.35 (d, J=8.2 Hz, 1 H, aromatic-H), 7.55 (d, J=8.2 Hz, 1 H, aromatic-H), 8.31 (brs, 1 H, NH). ¹³C-NMR (75 MHz, CDCl₃) δ 16 .2, 18.6, 20.8, 23.9, 23.9, 25.1, 27.5, 29.8, 33.4, 37, 37.9, 45.5, 47.2, 52.2, 53.1, 110.1 (aromatic-C), 111.2 (aromatic-C), 118.5 (aromatic-C), 119.66 (aromatic-C), 122.2 (aromatic-C), 122.5 (aromatic-C), 123.7 (aromatic-C), 123.9 (aromatic-C), 126.8 (aromatic-C), 127.6 (aromatic-C), 134.6 (aromatic-C), 136.1 (aromatic-C), 145.6 (aromatic-C), 146.9 (aromatic-C), 172.7 and 178.2 (COOCH₃ and CONH). HRMS m/z: calcd. for C₃₂H₄₁ N₂O₃ 501.3117 [M+1]⁺, found 501.3115.

Ethyl N-(abiet-8,11,13-trien-18-oyl) Glycyl-glycinate (14)

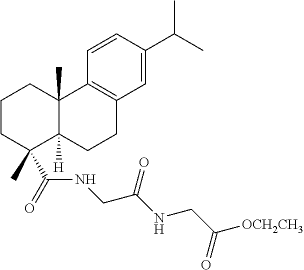 [SMILES: CCOC(=O)CNC(=O)CNC(=O)[C@]1(C)CCC[C@]2(C)c3ccc(C(C)C)cc3CC[C@@H]12]

Following the procedure for compound 3, compound 14 was prepared from 1 (500 mg; 1.66 mmol), EDC hydrochloride (478 mg; 2.49 mmol), HOBt monohydrate (338 mg; 2.49 mmol), H-Gly-Gly-OEt⁻HCl (472 mg; 2.40 mmol), and DIPEA (0.88 mL; 5.0 mmol), in DMF (5 mL). Compound 14: (600 mg, 81%). ¹H-NMR (300 MHz, CDCl₃) δ 1 .20 (s, 3 H), 1.21 (s, 3 H), 1.22 (s, 3 H), 1.27 (m, 3 H, CH₂CH₃), 1.31 (s, 3 H), 2.15 (m, 1 H), 2.29 (m, 1 H), 2.83 (m, 3 H), 4.01 (m, 4 H), 4.18 (m, 2 H), 6.75 (m, 1 H, NH), 6.85 (s, 1 H, aromatic-H), 6.98 (m, 2 H, aromatic-H and NH), 7.14 (m, 1 H, aromatic-H). ¹³C-NMR (75 MHz,

CDCl₃) δ 14.2, 16.5, 18.8, 21.3, 24, 25.3, 30, 33.5, 37.1, 37.3, 38, 41.4, 43.6, 45.5, 47.4, 61.6, 123.9 (aromatic-C), 124.1 (aromatic-C), 126.9 (aromatic-C), 134.7 (aromatic-C), 145.8 (aromatic-C), 147 (aromatic-C), 169.6, 169.7 and 179.5 (COOCH₃ and CONH). HRMS m/z: calcd. for C₂₆H₃₉N₂O₄ 443.2910 [M+1]⁺, found 443.2910.

Methyl N-(abiet-8,11,13-trien-18-oyl) L-alanyl-L-alanyl-L-alaninate (16)

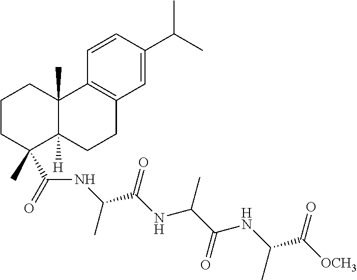 [SMILES: COC(=O)[C@H](C)NC(=O)C(C)NC(=O)[C@H](C)NC(=O)[C@]1(C)CCC[C@]2(C)c3ccc(C(C)C)cc3CC[C@@H]12]

Following the procedure for compound 3, compound 16 was prepared from 1 (250 mg; 0.83 mmol), EDC hydrochloride (239 mg; 1.25 mmol), HOBt monohydrate (230 mg; 1.25 mmol), H-Ala-Ala-Ala-OMe acetate salt (294 mg; 1.2 mmol), and DIPEA (0.44 mL; 2.5 mmol), in DMF (2.5 mL). Compound 16: (424 mg). The compound was purified by FCC with n-hexane: ethyl acetate (0 to 100%) and then dichloromethane: methanol (0 to 100%) to afford a white solid (375 mg; 85%). ¹H-NMR (300 MHz, CDCl₃) δ 1.20 (s, 3 H), 1.22 (s, 6 H), 1.29 (s, 3 H), 1.38 (m, 9 H), 2.12 (m, 1 H), 2.30 (m, 1 H), 2.84 (m, 3 H), 3.72 (s, 3 H, OCH₃), 4.53 (m, 3 H), 6.40 (m, 1 H, NH), 6.73 (m, 1 H, NH), 6.86 (m, 2 H, aromatic-H and NH), 6.98 (m, 1 H, aromatic-H), 7.15 (m, 1 H, aromatic-H). ¹³C-NMR (75 MHz, CDCl₃) δ 16.5, 18.2, 18.3, 18.4, 18.8, 21.3, 24.1, 24.1, 25.3, 30.1, 33.6, 37.2, 37.4, 38.1, 45.6, 47.3, 48.2, 49, 49.1, 52.5, 124 (aromatic-C), 124.1 (aromatic-C), 127 (aromatic-C), 134.6 (aromatic-C), 145.9 (aromatic-C), 146.9 (aromatic-C), 171.5, 172.5, 173.2 and 178.7 (COOCH₃ and CONH). HRMS m/z: calcd. for C₃₀H₄₆N₃O₅ 528.3437 [M+1]⁺, found 528.3448.

N-(abiet-8,11,13-trien-18-oyl) Glycine (17)

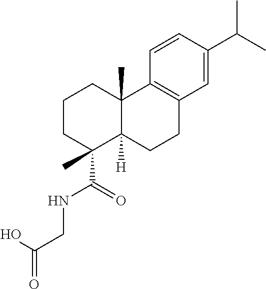 [SMILES: CC(C)c1ccc2c(c1)CC[C@H]1[C@](C)(C(=O)NCC(=O)O)CCC[C@]21C]

Compound 2 (200 mg, 0.54 mmol) was dissolved in THF:MeOH 1:1 (4.8 mL), at 0° C., under magnetic stirring. A 4 M solution of NaOH (4.4 mL) was added dropwise and after the addition the mixture was left to agitate at room temperature for 1 hour, after which the reaction was suspended by careful addition of aqueous 4 M HCl dropwise until the pH reached 6-7. The mixture was concentrated under vacuum and extracted with diethylether (3×75 mL) after the addition of water (25 mL).The resulting organic phase was washed with aqueous HCl (50 mL), water (50 mL), and brine (50 mL), dried with Na₂SO₄, filtered, and evaporated to dryness to afford 17 as a white solid (183 mg, 95%). Mp 181-182° C. IR (ATR) 3380, 1730, 1641, 1522, 1197, 820 cm⁻¹. ¹H-NMR (300 MHz, CDCl₃) δ 1.20 (s, 3 H), 1.23 (s, 6 H), 1.32 (s, 3 H), 2.13 (dd, J₁=12.4 Hz and J₂=2.1 Hz, 1 H), 2.31 (d, J=12.6 Hz, 1 H), 2.84 (m, 3 H), 4.06 (d, J₁=5 Hz and J₂=2.9 Hz, 2 H, —NHCH₂), 6.45 (brs, 1 H, NH), 6.87 (s, 1 H, 14-H), 6.99 (d, J₁=8.2 Hz and J₂=1.8 Hz, 1 H, aromatic-H), 7.15 (d, J=8.2 Hz, 1 H, aromatic-H), 8.06 (s, 1 H, OH). ¹³C-NMR (75 MHz, CDCl₃) δ 16.6, 18.8, 21.3, 24.1, 25.4, 30.1, 33.6, 37.2, 37.3, 38.1, 42.1, 45.7, 47.6, 124.1 (aromatic-C), 124.2 (aromatic-C), 127.1 (aromatic-C), 134.8 (aromatic-C), 145.9 (aromatic-C), 147 (aromatic-C), 173.4 and 180 (COOH and CONH). HRMS m/z: calcd. for C₂₂H₃₂NO₃ 358.2382 [M+1]⁺, found 358.2383.

N-(Abiet-8,11,13-trien-18-oyl) L-valine (18)

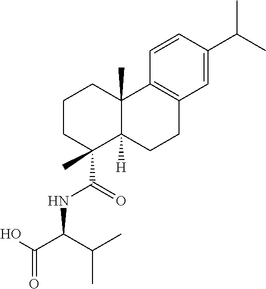 [SMILES: CC(C)c1ccc2c(c1)CC[C@H]1[C@](C)(C(=O)N[C@H](C(=O)O)C(C)C)CCC[C@]21C]

Following the procedure for compound 17, compound 18 was prepared from 4 (118 mg; 0.29 mmol), using THF:MeOH (4 mL), and 4 M NaOH (2.4 mL). Compound 18: (108 mg, 94%). Mp 149-151° C. IR (ATR) 3435, 3076, 1724, 1637, 1525, 1406, 1207, 821, 634 cm⁻¹. ¹H-NMR (300 MHz, CDCl₃) δ 0.98 (dd, J₁=10.8 Hz and J₂=6.9 Hz, 6 H, —CH(CH₃)₂), 1.21 (s, 3 H), 1.24 (s, 3 H), 1.25 (s, 3 H), 1.32 (s, 3 H), 2.11 (d, J₁=12.3 Hz, 1 H), 2.28 (m, 2 H), 2.85 (m, 3 H), 4.58 (m, 1 H, —NHCH—), 6.27 (d, J=8.2 Hz, 1 H, NH), 6.88 (s, 1 H, 14-H), 7.0 (d, J₁=8.2 Hz, 1 H, aromatic-H), 7.17 (d, J₁=8.2 Hz, 1 H, aromatic-H). ¹³C-NMR (75 MHz, CDCl₃) δ 16.3, 17.8, 18.7, 19.1, 21.2, 23.9, 23.9, 25.3, 29.9, 30.7, 33.4, 37.1, 37.4, 37.9, 45.6, 47.5, 57.3, 123.9 (aromatic-C), 124 (aromatic-C), 126.9 (aromatic-C), 134.6 (aromatic-C), 145.7 (aromatic-C), 146.8 (aromatic-C), 175.9 and 178.9 (COOH and CONH). HRMS m/z: calcd. for C₂₅H₃₈NO₃ 400.2852 [M+1]⁺, found 400.2852.

N-(abiet-8,11,13-trien-18-oyl) Ethyl-L-glycine (19)

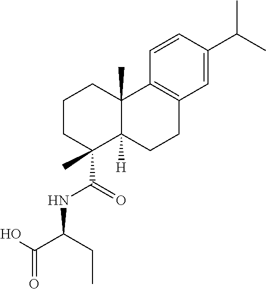 [SMILES: CC[C@H](NC(=O)[C@]1(C)CCC[C@]2(C)c3ccc(C(C)C)cc3CC[C@@H]12)C(=O)O]

Following the procedure for compound 17, compound 19 was prepared from 5 (150 mg; 0.38 mmol), using THF:MeOH (3.7 mL), and 4 M NaOH (3 mL). Compound 19: (133 mg, 92%). Mp 181-183° C. IR (ATR) 3435, 1716, 1623, 1529, 1224, 821 cm⁻¹. ¹H-NMR (300 MHz, CDCl₃) δ 0.97 (t, J=7.4 Hz, 3 H, —CH₂CH₃), 1.21 (s, 3 H), 1.23 (s, 3 H), 1.24 (s, 3 H), 1.31 (s, 3 H), 2.11 (dd, J₁=12.4 Hz and J₂=2 Hz, 1 H), 2.33 (d, J=12.1 Hz, 1 H), 2.85 (m, 3 H), 4.55 (dd, J₁=7.2 Hz and J₂=5.5 Hz, 1 H, —NHCH—), 6.29 (d, J=7.2 Hz, 1 H, NH), 6.88 (s, 1 H, 14-H), 7.0 (dd, J₁=8.2 Hz and J₂=1.8 Hz, 1 H, aromatic-H), 7.17 (d, J=8.2 Hz, 1 H, aromatic-H). ¹³C-NMR (75 MHz, CDCl₃) δ 9.7, 16.3, 18.6, 21.1, 23.9, 23.9, 25, 25.2, 29.9, 33.4, 37.1, 37.3, 37.9, 45.6, 47.3, 53.6, 123.9 (aromatic-C), 124 (aromatic-C), 126.9 (aromatic-C), 134.5 (aromatic-C), 145.7 (aromatic-C), 146.7 (aromatic-C), 176.1 and 179.1 (COOH and CONH). HRMS m/z: calcd. for C₂₄H₃₆NO₃ 386.2695 [M+1]⁺, found 386.2691.

N-(Abiet-8,11,13-trien-18-oyl) L-leucine (20)

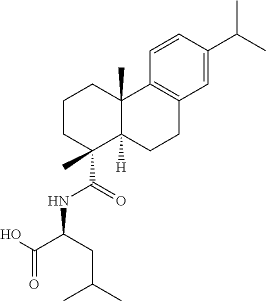 [SMILES: CC(C)C[C@H](NC(=O)[C@]1(C)CCC[C@]2(C)c3ccc(C(C)C)cc3CC[C@@H]12)C(=O)O]

Following the procedure for compound 17, compound 20 was prepared from 6 (150 mg; 0.35 mmol), using THF:MeOH (3.7 mL), and 4 M NaOH (3 mL). Compound 20 (120 mg, 83%). Mp 84-86° C. IR (ATR) 3359, 1733, 1627, 1523, 1232, 819 cm⁻¹. ¹H-NMR (300 MHz, CDCl₃) δ 0.96 (d, J=6 Hz, 6 H, —CH₂CH(CH₃)₂), 1.21 (s, 3 H), 1.24 (s, 6 H), 1.30 (s, 3 H), 2.09 (dd, J₁=12.3 Hz and J₂=1.7 Hz, 1 H), 2.33 (d, J=12.8 Hz, 1 H), 2.85 (m, 3 H), 4.60 (m, 1 H, —NHCH—), 6.12 (d, J=7.7 Hz, 1 H, NH), 6.88 (s, 1 H, 14-H), 7.0 (d, J₁=8.2 Hz, 1 H, aromatic-H), 7.17 (d, J=8.2 Hz, 1 H, aromatic-H), 10.49 (s, 1 H, OH). ¹³C-NMR (75 MHz, CDCl₃) δ 16.3, 18.6, 21.1, 21.7, 22.8, 23.9, 23.9, 25, 25.2, 29.8, 33.4, 37.1, 37.3, 37.9, 40.8, 45.7, 47.3, 51.1, 123.9 (aromatic-C), 124 (aromatic-C), 126.9 (aromatic-C), 134.6 (aromatic-C), 145.7 (aromatic-C), 146.8 (aromatic-C), 177.1 and 179.1 (COOH and CONH). HRMS m/z: calcd. for C₂₆H₄₀NO₃ 414.3008 [M+1]⁺, found 414.3007.

N-(Abiet-8,11,13-trien-18-oyl) L-phenylalanine (21)

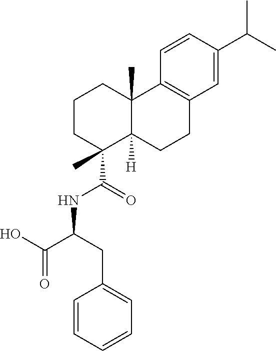 [SMILES: CC(C)c1ccc2c(c1)CC[C@H]1[C@](C)(C(=O)N[C@@H](Cc3ccccc3)C(=O)O)CCC[C@]21C]

Following the procedure for compound 17, compound 21 was prepared from 7 (150 mg; 0.32 mmol), using THF:MeOH (3.7 mL), and 4 M NaOH (3 mL). Compound 21 (135 mg, 93%). Mp 167-169° C. IR (ATR) 3442, 1755, 1598, 1537, 1261, 1091, 1020, 798 cm⁻¹. ¹H-NMR (300 MHz, DMSO-d₆) δ 1.10 (s, 3 H), 1.15 (s, 3 H), 1.20 (s, 3 H), 1.22 (s, 3 H), 2.08 (d, J=10.9 Hz, 1 H), 2.28 (d, J=12.3 Hz, 1 H), 2.80 (m, 3 H), 3 (ddd, J₁=24.4 Hz, J₂=13.8 Hz, and J₃=7.6 Hz, 2 H, —CH₂Ph), 4.54 (m, 1 H, —NHCH—), 6.87 (d, J=1.2 Hz, 1 H, aromatic-H), 7.01 (d, J₁=8.2 Hz, 1 H, aromatic-H), 7.26 (m, 6 H, aromatic-H), 7.80 (m, 1 H, NH), 12.55 (s, 1 H, OH). ¹³C-NMR (75 MHz, DMSO-d₆) δ 16.7, 18.9, 20.5, 24.3, 29.7, 33.3, 36.3, 36.6, 37, 37.7, 44.8, 46.7, 54, 123.9 (aromatic-C), 124.4 (aromatic-C), 126.6 (aromatic-C), 126.8 (aromatic-C), 128.4 (aromatic-C), 129.5 (aromatic-C), 135 (aromatic-C), 138.7 (aromatic-C), 145.4 (aromatic-C), 147.6 (aromatic-C), 173.9 and 177.8 (COOH and CONH). HRMS m/z: calcd. for C₂₉H₃₈NO₃ 448.2852 [M+1]⁺, found 448.2851.

N-(Abiet-8,11,13-trien-18-oyl) Cyclohexyl-L-alanine (22)

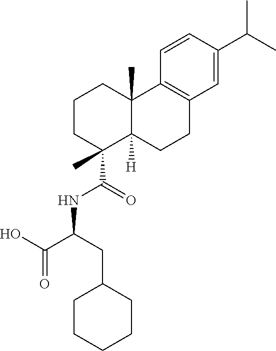 [SMILES: CC(C)c1ccc2c(c1)CC[C@H]1[C@](C)(C(=O)N[C@@H](CC3CCCCC3)C(=O)O)CCC[C@]21C]

Following the procedure for compound 17, compound 22 was prepared from 8 (100 mg; 0.21 mmol), using THF:MeOH (3 mL), and 4 M NaOH (1.8 mL). Compound 22: (95 mg, 98%). Mp 130-132° C. IR (ATR) 3346, 1732, 1625, 1525, 1232, 819 cm⁻¹. ¹H-NMR (300 MH, CDCl₃) δ 1.17 (s, 3 H), 1.24 (s, 6 H), 1.31 (s, 3 H), 2.09 (m, 1 H), 2.33 (d, J=11.8 Hz, 1 H), 2.86 (m, 3 H), 4.64 (m, 1 H, —NHCH—), 6.11 (d, J=7.7 Hz, 1 H, NH), 6.89 (s, 1 H, 14-H), 7.0 (dd, J₁=8.2 Hz and J₂=1.7 Hz, 1 H, aromatic-H), 7.17 (d, J=8.2 Hz, 1 H, aromatic-H). ¹³C-NMR (75 MHz, CDCl₃) δ 16.3, 18.6, 21.1, 23.9, 23.9, 25.2, 26, 26.2, 26.3, 29.8, 32.3, 33.4, 33.5, 34.3, 37.1, 37.1, 37.9, 39.2, 45.7, 47.3, 50.4, 123.9 (aromatic-C), 124 (aromatic-C), 126.9 (aromatic-C), 134.5 (aromatic-C), 145.7 (aromatic-C), 146.8 (aromatic-C), 176.9 and 179.1 (COOH and CONH). HRMS m/z: calcd. for C₂₉H₄₄NO₃ 454.3321 [M+1]⁺, found 454.3322.

N-(Abiet-8,11,13-trien-18-oyl) D-methionine (23)

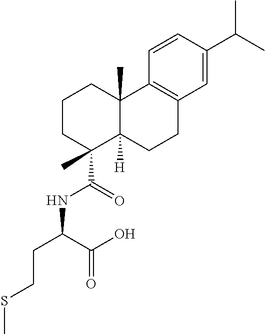 [SMILES: CSCC[C@@H](NC(=O)[C@]1(C)CCC[C@]2(C)c3ccc(C(C)C)cc3CC[C@@H]12)C(=O)O]

Following the procedure for compound 17, compound 23 was prepared from 9 (250 mg; 0.56 mmol), using THF:MeOH (7.9 mL), and 4 M NaOH (4.7 mL). Compound 23: (234 mg, 96%). Mp 84-86° C. IR (ATR) 3373, 1737, 1631, 1541, 1228, 821 cm⁻¹. ¹H-NMR (300 MHz, CDCl₃) δ 1.21 (s, 3 H), 1.24 (s, 6 H), 1.32 (s, 3 H), 2.10 (s, 3 H, SCH₃), 2.33, (m, 1 H), 2.36 (t, J=7.3 Hz , 2 H), 2.85 (m, 3 H), 4.72 (m, 1 H, —NHCH—), 6.70 (d, J=7.2 Hz, 1 H, NH), 6.88 (d, J=1.6 Hz, 1 H, 14-H), 7.0 (dd, J₁=8.2 Hz and J₂=1.8 Hz, 1 H, aromatic-H), 7.17 (d, J=8.2 Hz, 1 H, aromatic-H). ¹³C-NMR (75 MHz, CDCl₃) δ 15.6, 16.5, 18.8, 21.2, 24.1, 25.3, 30.1, 30.2, 30.8, 33.6, 37.2, 37.3, 38, 45.8, 47.5, 52.3, 124 (aromatic-C), 124.1 (aromatic-C), 127 (aromatic-C), 134.6 (aromatic-C), 145.9 (aromatic-C), 146.9 (aromatic-C), 175.6 and 179.7 (COOH and CONH). HRMS m/z: calcd. for C₂₅H₃₈NO₃S 432.2573 [M+1]⁺, found 432.2573.

N-(Abiet-8,11,13-trien-18-oyl) D-tyrosine (24)

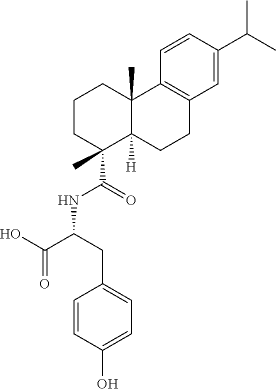 [SMILES: CC(C)c1ccc2c(c1)CC[C@H]1[C@](C)(C(=O)N[C@H](Cc3ccc(O)cc3)C(=O)O)CCC[C@]21C]

Following the procedure for compound 17, compound 24 was prepared from 10 (250 mg; 0.52 mmol), using THF:MeOH (7.3 mL), and 4 M NaOH (4.4 mL). Compound 24: (217 mg, 89%). Mp 108-110° C. IR (ATR) 3280, 1718, 1616, 1515, 1220, 821 cm⁻¹. ¹H-NMR (300 MHz, CDCl₃) δ 1 .20 (s, 3 H), 1.21 (s, 3 H), 1.23 (s, 3 H), 1.24 (s, 3 H), 2.08 (dd, J₁=11.1 and J₂=2 Hz, 1 H), 2.30 (d, J=12.6 Hz, 1 H), 2.80 (m, 3 H), 3.07 (m, 2 H), 4.87 (m, 1 H, —NHCH—), 6.37 (d, J=7.5 Hz, 1 H, NH), 6.72 (d, 1 H, J=8.2 Hz, aromatic-H), 6.86 (s, 1 H, aromatic-H), 6.98 (m, 3 H, aromatic-H), 7.15 (d, J=8.2 Hz, 1 H, aromatic-H), 7.27 (brs, OH). ¹³C-NMR (75 MHz, CDCl₃) δ 16.2, 18.6, 21, 23.9, 23.9, 25.2, 29.8, 33.4, 36.6, 37, 37, 37.8, 45.3, 47.4, 53.5, 115.7 (aromatic-C), 123.8 (aromatic-C), 124 (aromatic-C), 126.9 (aromatic-C), 127.1 (aromatic-C), 130.2 (aromatic-C), 134.5 (aromatic-C), 145.7 (aromatic-C), 146.7 (aromatic-C), 155.2 (aromatic-C), 175.1 and 179.5 (COOH and CONH). HRMS m/z: calcd. for C₂₉H₃₈NC₄ 464.2801 [M+1]⁺, found 464.2801.

N-(Abiet-8,11,13-trien-18-oyl) D-tryptophan (25)

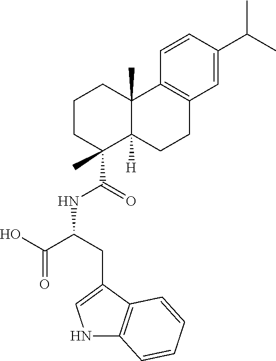 [SMILES: CC(C)c1ccc2c(c1)CC[C@H]1[C@](C)(C(=O)N[C@H](Cc3c[nH]c4ccccc34)C(=O)O)CCC[C@]21C]

Following the procedure for compound 17, compound 25 was prepared from 11 (250 mg; 0.50 mmol), using THF:MeOH (5.0 mL), and 4 M NaOH (4.2 mL). Compound 25: (226 mg, 93%). Mp 118-120° C. IR (ATR) 3402, 3257, 1728, 1629, 1529, 740 cm⁻¹. ¹H-NMR (300 MHz, CDCl₃) δ 1.05 (s, 3 H), 1.13 (s, 3 H), 1.22 (s, 3 H), 1.24 (s, 3 H), 1.89 (d, J=12.3 Hz, 1 H), 2.23 (d,

J=12.6 Hz, 1 H), 3.36 (m, 2 H, —NHCHCH₂—), 4.83 (m, 1 H, —NHCH—), 6.25 (m, 1 H, NH), 6.79 (m, 1 H, aromatic-H), 7.07 (m, 5 H, aromatic-H), 7.29 (m, 2 H, aromatic-H), 7.56 (d, J=7.8 Hz, 1 H, aromatic-H), 8.11 (s, 1 H, aromatic NH). ¹³C-NMR (75 MHz, CDCl₃) δ 16.1, 18.5, 20.8, 23.9, 23.9, 25.1, 26.8, 29.6, 33.4, 36.8, 36.9, 37.8, 45.5, 47.3, 53.6, 109.5 (aromatic-C), 111.4 (aromatic-C), 118.3 (aromatic-C), 122.2 (aromatic-C), 123.1 (aromatic-C), 123.8 (aromatic-C), 123.9 (aromatic-C), 126.8 (aromatic-C), 134.6 (aromatic-C), 136.1 (aromatic-C), 145.6 (aromatic-C), 146.7 (aromatic-C), 175.3 and 179.7 (COOH and CONH). HRMS m/z: calcd. for C₃₁H₃₉N₂O₃ 487.2961 [M+1]⁺, found 487.2961.

N-(Abiet-8,11,13-trien-18-oyl) Glycyl-glycine (27)

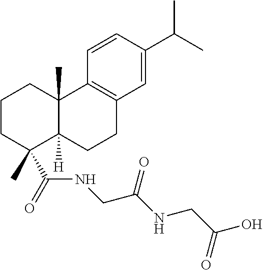 [SMILES: CC(C)c1ccc2c(c1)CC[C@H]1[C@](C)(C(=O)NCC(=O)NCC(=O)O)CCC[C@]21C]

Following the procedure for compound 17, compound 27 was prepared from 14 (600 mg; 1.35 mmol), using THF:MeOH (12 mL), and 4 M NaOH (11 mL). Compound 27: (270 mg, 48%). ¹H-NMR (300 MHz, CDCl₃) δ 1.19 (s, 3 H), 1.22 (s, 3 H), 1.28 (s, 3 H), 2.12 (m, 1 H), 2.27 (m, 1 H), 2.81 (m, 3 H), 3.99 (m, 4 H), 6.85 (m, 1 H, aromatic-H), 6.98 (m, 1 H, aromatic-H), 7.13 (m, 2 H, aromatic-H and NH), 7.29 (NH), 8.86 (brs, 1 H, OH). ¹³C-NMR (75 MHz, CDCl₃) δ 16.5, 18.7, 21.3, 24.1, 25.3, 30, 31.7, 33.6, 37.1, 37.2, 38, 41.5, 43.4, 45.4, 47.5, 124.1 (aromatic-C), 127 (aromatic-C), 134.6 (aromatic-C), 145.8 (aromatic-C), 146.9 (aromatic-C), 170.4, 172.3 and 180.4 (COOH and CONH). HRMS m/z: calcd. for C₂₄H₃₅N₂O₄ 415.2597 [M+1]⁺, found 415.2597.

Methyl N-(7-oxo-abiet-8,11,13-trien-18-oyl) Glycinate (28)

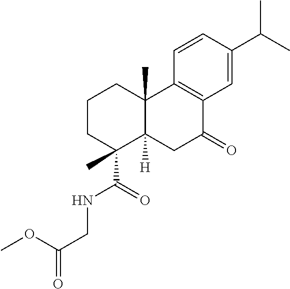 [SMILES: COC(=O)CNC(=O)[C@]1(C)CCC[C@]2(C)c3ccc(C(C)C)cc3C(=O)C[C@@H]12]

Compound 3 (200 mg, 0.54 mmol) was dissolved in glacial acetic acid (3.1 mL) and added dropwise to a cooled (0° C.) solution of chromium (VI) oxide (60 mg, 0.60 mmol) in glacial acetic acid (0.8 mL) and ethyl acetate (1.7 mL), over a period of about 10 minutes. The reaction mixture was then warmed to 50° C., under argon. After 4 hours more chromium (VI) oxide (60 mg, 0.60 mmol) was added and after 1 hour the reaction was completed. The reaction was suspended by cooling in an ice bath and adding ice and dichloromethane (150 mL). The aqueous phase was further extracted with dichloromethane (2×75 mL). The resulting organic phase was washed with water (50 mL), saturated NaHCO₃ solution (50 mL), water (50 mL), and brine (50 mL), dried with Na₂SO₄, filtered, and evaporated to dryness. Purification by FCC using ethyl acetate:n-hexane (2:1) afforded 28 as a white solid (116 mg, 56%). Mp 65-67° C. IR (ATR) 3392, 1755, 1678, 1645, 1526, 1198 cm⁻¹. ¹H-NMR (300 MHz, CDCl₃) δ 1.22 (d, J=1 Hz, 3 H), 1.24 (d, J=1 Hz, 3 H), 1.26 (s, 3 H), 1.39 (s, 3 H), 2.35 (d, J=11.7 Hz, 1 H), 2.58 (m, 3 H), 2.91 (m, 1 H), 3.76 (s, 3 H, OCH₃), 4.01 (m, 2 H, —NHCH₂), 6.33 (brs, 1 H, NH), 7.27 (d, J=8.2 Hz, 1 H, aromatic-H), 7.39 (dd, J₁=8.6 Hz and J₂=1.2 Hz, 1 H, aromatic-H), 7.83 (d, J=1.2 Hz, 1 H, 14-H). ¹³C-NMR (75 MHz, CDCl₃) δ 16.5, 18.4, 23.9, 24, 33.8, 36.9, 37.2, 37.4, 37.5, 41.8, 44.6, 46.6, 52.6, 123.5 (aromatic-C), 125.2 (aromatic-C), 130.9 (aromatic-C), 132.7 (aromatic-C), 147.1 (aromatic-C), 153.2 (aromatic-C), 170.7 and 177.5 (COOCH₃ and CONH), 198.7 (C7). HRMS m/z: calcd. for C₂₃H₃₂NO₃ 386.2331 [M+1]⁺, found 386.2333.

Methyl N-(7-oxo-abiet-8,11,13-trien-18-oyl) Cyclohexyl-L-alaninate (29)

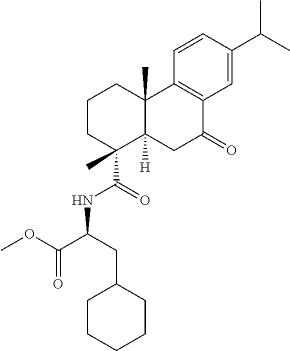 [SMILES: COC(=O)[C@H](CC1CCCCC1)NC(=O)[C@]1(C)CCC[C@]2(C)c3ccc(C(C)C)cc3C(=O)C[C@@H]12]

Following the procedure for compound 28, compound 29 was prepared from 8 (200 mg, 0.43 mmol), chromium oxide (133 mg, 1.34 mmol), glacial acetic acid (3.9 mL), and ethyl acetate (1.7 mL). Compound 29: (98 mg, 47%). Mp 66-68° C. IR (ATR) 3394, 1745, 1681, 1676, 1521, 1450, 1251, 1197, 835 cm⁻¹. ¹H-NMR (CDCl₃) δ 1.23 (s, 3 H), 1.25 (s, 3 H), 1.27 (s, 3 H), 1.39 (s, 3 H), 2.38 (m, 2 H), 2.67 (m, 2 H), 2.92 (m, 1 H), 3.72 (s, 3 H, OCH₃), 4.64 (d, J₁=14 Hz and J₂=8.4, 1 H, —NHCH—), 6.10 (d, J=8.1 Hz, 1 H, NH), 7.28 (d, J=8.1 Hz, aromatic-H), 7.39 (d, J=8.1 Hz, aromatic-H), 7.85 (s, 1 H, 14-H). ¹³C-NMR (75 MHz, CDCl₃) δ 16.5, 18.4, 23.8, 23.9, 23.9, 26.1, 26.3, 26.5, 32.7, 33.6, 33.7, 34.6, 37.1, 37.2, 37.4, 37.4, 40.2, 44, 46.5, 50.5, 52.4, 123.4 (aromatic-C), 125.2 (aromatic-C), 131 (aromatic-C), 132.5 (aromatic-C), 147 (aromatic-C), 153.1 (aromatic-C), 173.8 and 176.9 (COOCH₃ and CONH), 198.4 (C7). HRMS m/z: calcd. for C₃₀H₄₄NO₃ 482.3270 [M+1]⁺, found 482.3271.

Methyl N-(abiet-8,11,13-trien-18-oyl) D-phenylalaninate (30).

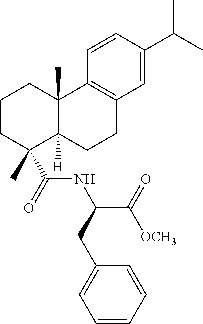 [SMILES: COC(=O)[C@@H](Cc1ccccc1)NC(=O)[C@]1(C)CCC[C@]2(C)c3ccc(C(C)C)cc3CC[C@@H]12]

Compound 1 (1.00 g, 3.33 mmol), D-phenylalanine methyl ester hydrochloride (1.08 g, 5.00 mmol), EDC (0.96 g, 5.0 mmol), and HOBt (0.68 g, 5.0 mmol) were dissolved in dry DMF (11 mL). The reaction mixture was stirred until all the solids were dissolved. DIPEA (1.74 mL, 10.0 mmol) was added. After stirring the mixture at room temperature for 105 min, it was poured into water (100 mL). The resulting precipitate was filtered and purified by FCC (silica column, 15→>25% EtOAc in n-hexane). Compound 30: white solid (0.65 g, 42%). ¹H NMR (300 MHz, CDCl₃) δ ppm 1.19 (m, 9 H), 1.22 (m, 3 H), 2.04 (d, J=12.4 Hz, 1 H), 2.28 (d, J=13.5 Hz, 1 H), 2.76 (m, 3 H), 3.05 (dd, J=14.1, 6.4 Hz, 1 H), 3.17 (dd, J=14.1, 5.3 Hz, 1 H), 3.73 (s, 3 H), 4.88 (m, 1 H), 6.15 (d, J=7.6 Hz, 1 H), 6.83 (s, 1 H), 6.97 (d, J=8.2 Hz, 1 H), 7.10 (m, 3 H), 7.25 (m, 3 H). ¹³C NMR (75 MHz, CDCl₃) δ ppm 16.5, 18.9, 21.2, 24.1, 24.1, 25.4, 30.0, 33.6, 37.2, 37.4, 38.0, 38.1, 45.6, 47.5, 52.4, 53.3, 124.0, 124.2, 127.0, 127.3, 128.7, 129.3, 134.8, 136.2, 145.8, 147.1, 172.6, 178.1. IR (ATR) 3360, 1742, 1639, 1497, 1213, 700. HRMS m/z: calcd. for C₃₀1-1₄₀NO₃ [M+H]⁺462.3008, found 462.3009.

N-(Abiet-8,11,13-trien-18-oyl) D-phenylalanine (31).

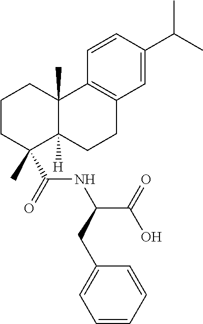 [SMILES: CC(C)c1ccc2c(c1)CC[C@H]1[C@](C)(C(=O)N[C@H](Cc3ccccc3)C(=O)O)CCC[C@]21C]

Compound 30 550 mg, 1.2 mmol) was dissolved in 1:1 THF/MeOH (15 mL). A 4 M aqueous solution of NaOH (13 mL) was added. After stirring the mixture at room temperature for 3 h, it was cooled on an ice bath and acidified with 4 M HCl. The precipitate was filtered and dried in vacuo. Compound 31: white solid (490 mg, 91%). ¹H NMR (300 MHz, CDCl₃) δ 1.19 (s, 6 H), 1.22 (s, 3 H), 1.24 (s, 3 H), 2.03 (d, J=12.3 Hz, 1 H), 2.29 (d, J=12.3 Hz, 1 H), 2.78 (m, 3 H), 3.11 (dd, J=14.1, 7.0 Hz, 1 H), 3.29 (dd, J=14.1, 5.8 Hz, 1 H), 4.87 (q, J=6.4 Hz, 1 H), 6.17 (d, J=7.0 Hz, 1 H), 6.85 (s, 1 H), 6.99 (d, J=8.2 Hz, 1 H), 7.16 (m, 3 H), 7.27 (m, 3 H). ¹³C NMR (75 MHz, CDCl₃) δ 16.4, 18.8, 21.2, 24.2, 25.3, 29.9, 33.6, 37.2, 37.2, 37.3, 38.0, 45.6, 47.6, 53.5, 124.0, 124.1, 127.1, 127.4, 128.9, 129.4, 134.8, 135.9, 145.9, 146.9, 175.1, 179.5. IR (ATR) 3445, 1747, 1600, 1539, 1205, 700 cm⁻¹. HRMS m/z: calcd. for C₂₉H₃₈NO₃ [M+H]⁺448.2852, found 448.2856.

N-(7-Oxoabiet-8,11,13-trien-18-oyl) Cyclohexyl-L-alanine (32).

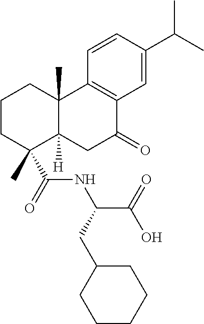 [SMILES: CC(C)c1ccc2c(c1)C(=O)C[C@H]1[C@](C)(C(=O)N[C@@H](CC3CCCCC3)C(=O)O)CCC[C@]21C]

Compound 29 350 mg, 0.73 mmol) was dissolved in THF/MeOH 1:1 (10 mL) and a 4 M aqueous solution of NaOH (8.5 mL) was added. The color of the reaction mixture changed to yellow. The reaction mixture was then stirred at room temperature for 2 h 45 min. The mixture was acidified with a 4 M aqueous solution of HCl and concentrated. Water was added and the mixture was extracted three times with ethyl acetate. The organic phase was then washed with a 1 M aqueous solution of HCl, water and brine and dried with Na₂SO₄ and evaporated. The crude product was purified by FCC (silica column, 50% EtOAc in n-hexane and 2% acetic acid). Compound 32: white solid (0.23 g, 68%). ¹H NMR (300 MHz, CDCl₃) δ 1.23 (s, 3 H), 1.25 (s, 3 H), 1.26 (s, 3 H), 1.38 (s, 3 H), 2.40 (m, 2 H), 2.65 (m, 2 H), 2.92 (sept, J=6.9 Hz, 1 H), 4.62 (m, 1 H), 6.15 (d, J=7.6 Hz, 1 H), 7.28 (d, J=9.4 Hz, 1 H), 7.40 (dd, J=8.2, 1.8 Hz, 1 H), 7.85 (d, J=2.3 Hz, 1 H), 9.21 (bs, 1 H). ¹³C NMR (75 MHz, CDCl₃) δ 16.5, 18.4, 23.9, 24.0, 24.0, 26.1, 26.3, 26.5, 32.6, 33.7, 33.7, 34.6, 37.0, 37.1, 37.4, 39.6, 43.9, 46.6, 50.6, 123.5, 125.3, 130.9, 132.8, 147.1, 153.2, 176.7, 177.8, 198.8. IR (ATR) 3348, 1732, 1681, 1636, 1531,1232, 1194, 833 cm⁻¹. HRMS m/z: calcd. for C₂₉H₄₁NO₄Na [M+Na]⁺490.2933, found 490.2932.

Methyl N-(abiet-8,11,13-trien-18-oyl) H-r3 (3-pyridyl)-D-alaninate (33).

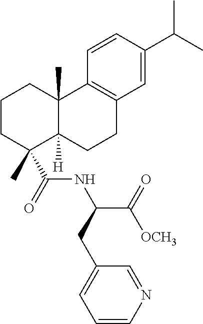 [SMILES: COC(=O)[C@@H](Cc1cccnc1)NC(=O)[C@]1(C)CCC[C@]2(C)c3ccc(C(C)C)cc3CC[C@@H]12]

Compound 1 (540 mg, 1.80 mmol), H-r3-(3-pyridyI)-b-Ala-OMe hydrochloride (0.500 g, 1.98 mmol), EDC (380 mg, 1.98 mmol), and HOBt (270 mg, 1.98 mmol) were dissolved in dry DMF (11 mL). DIPEA (1.74 mL, 10.0 mmol) was added. After stirring the mixture at room temperature for 16 h, it was poured into cold H₂O (80 mL). The precipitated solid was filtered and purified by FCC (silica column, 50% EtOAc in n-hexane) Compound 33: white solid (0.70 g, 84%). ¹H NMR (300 MHz, CDCl₃) δ 1.20 (s, 6 H), 1.23 (s, 3 H), 1.23 (s, 3 H), 2.09 (dd, J=12.3, 2.3 Hz, 1 H), 2.29 (d, J=11.7 Hz, 1 H), 2.80 (m, 3 H), 3.07 (dd, J=14.1, 6.5 Hz, 1 H), 3.24 (dd, J=14.1, 5.3 Hz, 1 H), 3.77 (s, 3 H), 4.93 (m, 1 H), 6.30 (d, J=7.0 Hz, 1 H), 6.86 (d, J=1.8 Hz, 1 H), 6.99 (dd, J=8.2, 2.3 Hz, 1 H), 7.15 (d, J=8.2 Hz, 1 H), 7.24 (m, 1 H), 7.49 (dt, J=8.2, 2.3 Hz, 1 H), 8.35 (d, J=1.8 Hz, 1 H), 8.51 (dd, J=5.3, 1.8 Hz, 1 H). ¹³C NMR (75 MHz, CDCl₃) δ ppm 16.5, 18.8, 21.3, 24.1, 25.4, 30.0, 33.6, 35.3, 37.2, 37.5, 38.0, 45.4, 47.5, 52.7, 53.0, 123.6, 124.0, 124.2, 127.0, 132.1, 134.7, 137.1), 145.9), 146.9, 148.3, 150.2, 172.1, 178.4. IR (ATR) 3252, 1732, 1653, 1539, 1234, 708. HRMS m/z: calcd. for C₂₉H₃₉N₂O₃ [M+H]⁺463.2961, found 463.2957.

Methyl N-(abiet-8,11,13-trien-18-oyl) H-(3-(3-pyridyl-N-oxide)-o-alaninate (34).

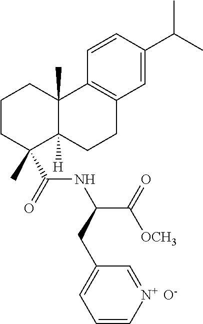

Compound 33 (0.10 g, 0.22 mmol) was dissolved in CHCl₃ (2 mL). This solution was cooled on an ice bath and m-CPBA (0.10 g, 0.45 mmol) was added in small portions. The reaction mixture was stirred at room temperature for 17 h. It was transferred to a silica gel column and purified by FCC (10% MeOH in EtOAc). Compound 34: white solid (50 mg, 47%). ¹H NMR (300 MHz, CDCl₃) δ 1.20 (s, 3 H), 1.20 (s, 3 H), 1.22 (s, 3 H), 1.26 (s, 3 H), 2.11 (dd, J=12.3, 2.1 Hz, 1 H), 2.29 (d, J=11.7 Hz, 1 H), 2.80 (m, 3 H), 3.03 (dd, J=14.4, 6.2 Hz, 1 H), 3.21 (dd, J=14.4, 5.6 Hz, 1 H), 3.79 (m, 3 H), 4.88 (q, J=6.5 Hz, 1 H), 6.48 (d, J=7.0 Hz, 1 H), 6.86 (s, 1 H), 6.99 (dd, J=8.2, 1.8 Hz, 1 H), 7.15 (d, J=8.2 Hz, 2 H), 7.24 (m, 1 H), 8.05 (s, 1 H), 8.14 (d, J=6.5 Hz, 1 H). ¹³C NMR (75 MHz, CDCl₃) δ 16.6, 18.8, 21.4, 24.1, 24.1, 25.4, 30.0, 33.6, 35.0, 37.2, 37.5, 38.0, 45.4, 47.6, 52.7, 53.0, 124.1, 124.2, 125.8, 127.0, 127.6, 134.6, 136.3, 137.9, 139.8, 145.9, 146.9, 171.6, 178.8. IR (ATR) 3254, 1742, 1649, 1261, 1213, 1159, 681 cm⁻¹. HRMS m/z: calcd. for C₂₉H₃₈N₂O₄Na [M+Na]⁺501.2729, found 501.2731.

Methyl N-(abiet-8,11,13-trien-18-oyl) H-(3-(3-pyridyI)-o-alanine (35).

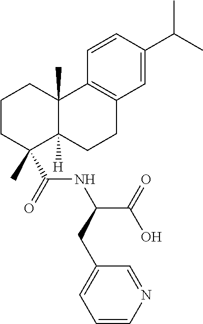 [SMILES: CC(C)c1ccc2c(c1)CC[C@H]1[C@](C)(C(=O)N[C@H](Cc3cccnc3)C(=O)O)CCC[C@]21C]

Following the procedure for compound 17, compound 35 was prepared from 33 (240 mg, 0.52 mmol), using THF:MeOH 1:1 (4.8 mL) and 4 M NaOH (4.3 mL). The reaction mixture was acidified with 1 M HCl and the aqueous phase was extracted with diethyl ether. The organic phase was dried with anhydrous Na₂SO₄ and evaporated. The crude product was purified by FCC (DCM/MeOH). Compound 35: white solid (102 mg, 44%). ¹H NMR (300 MHz, DMSO-d₆) δ 1.08 (s, 3 H), 1.09 (s, 3 H), 1.13 (s, 3 H), 1.16 (s, 3 H), 1.88 (d, J=11.7 Hz, 1 H), 2.25 (d, J=12.3 Hz, 1 H), 2.70 (m, 3 H), 3.03 (dd, J=13.5, 5.3 Hz, 1 H), 3.14 (dd, J=13.5, 5.3 Hz, 1 H), 4.14 (m, 1 H), 6.79 (m, 1 H), 6.94 (d, J=8.21 Hz, 1 H), 7.12 (m, 2 H), 7.21 (dd, J=7.62, 4.69 Hz, 1 H), 7.49 (d, J=7.62 Hz, 1 H), 8.30 (m, 1 H), 8.34 (dd, J=4.69, 1.76 Hz, 1 H). ¹³C-NMR (75 MHz, DMSO-d₆) δ 16.2, 18.3, 20.4, 23.9, 24.9, 29.3, 32.8, 34.0, 36.5, 36.7, 37.7, 44.8, 46.2, 54.5, 122.7, 123.6, 123.9, 126.3, 134.2, 134.2, 136.8, 144.9, 146.9, 150.4, 173.3, 176.1. IR (ATR) 3316, 1594, 1497, 1415, 821, 712 cm⁻¹. HRMS m/z: calcd. for C₂₈H₃₇N₂O₃449.2804 [M+H]⁺, found 449.2805.

Methyl N-(7-hydroxyiminoabiet-8,11,13-trien-18-oyl) Cyclohexyl-L-alaninate (36).

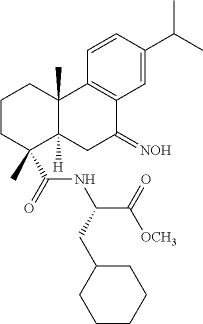

Compound 29 (250 mg, 0.52 mmol) and hydroxylamine hydrochloride (0.060 g, 0.88 mmol) were dissolved in EtOH (1.5 mL) and pyridine (63 μL) was added. The reaction mixture was stirred in a closed vial at 100° C. for 3 h. Solvents were evaporated and the residue was purified by FCC (silica column, 15→50% EtOAc in n-hexane). Compound 36: white solid (200 mg, 78%). ¹H NMR (300 MHz, CDCl₃) δ 1.12 (s, 3 H), 1.23 (s, 3 H), 1.25 (s, 3 H), 1.42 (s, 3 H), 2.27 (m, 2 H), 2.67 (m, 2 H), 2.88 (sept, J=6.9 Hz, 1 H), 3.71 (s, 3 H), 4.66 (m, 1 H), 6.13 (d, J=8.2 Hz, 1 H), 7.20 (m, 2 H), 7.68 (s, 1 H). ¹³C NMR (75 MHz CDCl₃) δ 16.6, 18.4, 23.1, 23.5, 23.9, 24.2, 26.0, 26.3, 26.5, 32.5, 33.7, 33.8, 34.4, 36.7, 37.3, 37.4, 40.1, 42.1, 46.5, 50.5, 52.3, 122.4, 122.9, 128.0, 129.0, 146.6, 148.8, 155.6, 174.0, 177.4. IR (ATR) 3360, 1738, 1641, 1508, 1447, 1204, 951, 729 cm⁻¹ HRMS m/z: calcd. for C₃₀H₄₅N₂O_{4 [M+H]}⁺497.3379, found 497.3379.

### REFERENCES

Donlan R M and Costerton J W. Biofilms; Survival mechanisms of clinically relevant microorganisms. Clinical Microbiology Rev. 2002 Apr;15(2):167-93.

Coenye T, Van Dijck P and Bjarnsholt T, Forsberg A. Microbial Biofilmr—The coming age of a research field. Pathol Dis. 2014 Apr;70(3):203-4.

Fallarero A, Skogman M, Kujala J, Rajaratnam M, Moreira V M, Yli-Kauhaluoma J and Vuorela P. (+)-Dehydroabietic Acid, an Abietane-Type Diterpene, Inhibits Staphylococcus aureus Biofilms in Vitro. Int. J. Mol. Sci. 2013, 14, 12054-12072; doi:10.3390/ijms140612054.

Kolodkin-Gal I, Romero D, Cao S, Clardy J, Kolter R, and Losick R. D-Amino Acids Trigger Biofilm Disassembly. Science, 2010, 328:627-629.

Hochbaum Al, Kolodkin-Gal I, Foulston L, Kolter R, Aizenberg J, and Losick R. Inhibitory Effects of D-Amino Acids on Staphylococcus aureus Biofilm Development. J Bacteriol., 2011, 193(20):5616-5622.

Blackledge M S, Melander C. Small molecule inhibition of bacterial two-component systems to combat antibiotic resistance and virulence. Future Med Chem. 2013 Jul;5(11):1265-84.

Blackledge M S, Worthington R J, Melander C. Biologically inspired strategies for combating bacterial biofilms. Curr Opin Pharmacol. 2013 Oct;13(5):699-706. Epub 2013 Jul 18.

Sandberg M E, Schellmann D, Brunhofer G, Erker T, Busygin I, Leino R, Vuorela P M, Fallarero A. Pros and cons of using resazurin staining for quantification of viable Staphylococcus aureus biofilms in a screening assay. J Microbiol Methods. 2009 Jul;78(1):104-6.

Rios, J. L., Recio, M. G. Medicinal plants and antimicrobial activity. J. Ethnopharmacol. 2005, 100, 80-84.

Worthington R J, Richards J J, Melander C. Small molecule control of bacterial biofilms. Org Biomol Chem. 2012 Oct 7;10(37):7457-74.

Pitts B, Hamilton M A, Zelver N, Stewart P S. A microtiter-plate screening method for biofilm disinfection and removal. J Microbiol Methods. 2003 Aug;54(2):269-76.

Okuda et al. Antimicrob Agents Chemother. 2013, 57(11):5572-9. Effects of Bacteriocins on Methicillin-Resistant Staphylococcus aureus Biofilm.

Karlsson, D., Fallarero, A., Brunhofer, G., Mayer, C., Prakash, O, Mohan, C. G., Vuorela, P., Erker, T. The exploration of thienothiazines as selective butyrylcholinesterase inhibitors. Eur J Pharm Sci. 2012 Aug 30;47(1):190-205.

Landini, P.; Antoniani, D.; Burgess, J.G.; Nijland, R. Molecular mechanisms of compounds affecting bacterial biofilm formation and dispersal. Appl. Microbiol. Biotechnol. 2010, 86, 813-823.

Toté, K.; Berghe, D. V.; Deschacht, M.; de Wit, K.; Maes, L.; Cos, P. Inhibitory efficacy of various antibiotics on matrix and viable mass of Staphylococcus aureus and Pseudomonas aeruginosa biofilms. Int. J. Antimicrob. Agents 2009, 33, 525-531.

Skogman, M. E.; Vuorela, P. M.; Fallarero, A. Combining biofilm matrix measurements with biomass and viability assays in susceptibility assessments of antimicrobials against Staphylococcus aureus biofilms. J. Antibiot. 2012, 65, 453-459.

Kuz\acute{m}a, /L.; Rózalski, M.; Walencka, E.; Rózalska, B.; Wysokińska, H. Antimicrobial activity of diterpenoids from hairy roots of Salvia sclarea I.: Salvipisone as a potential anti-biofilm agent active against antibiotic resistant Staphylococci. Phytomedicine 2007, 14, 31-35

Zou J, Pan L, Li Q, Pu J, Yao P, Zhu M, Banas J A, Zhang H, Sun H. Rubesanolides C-E: abietane diterpenoids isolated from Isodon rubescens and evaluation of their anti-biofilm activity. Org Biomol Chem. 2012 Jul 14;10(26):5039-44.

Tsuchiya, Tomofusa; Shiota, Sumiko From PCT Int. Appl. (2010), WO 2010119638 A1 20101021. I Language: Japanese, Database: CAPLUS Biofilm formation inhibitor containing diterpenoid or rosin, and use thereof.

Ali, F.; Sangwan, P. L.; Koul, S.; Pandey, A.; Bani, S.; Abdullah, S. T.; Sharma, P. R.; Kitchlu, S.; Khan, I. A. 4-epi-pimaric acid: A phytomolecule as a potent antibacterial and anti-biofilm agent for oral cavity pathogens. Eur. J. Clin. Microbiol. Infect. Dis. 2012, 31, 149-159.

1. A compound of formula (I), or a pharmaceutically acceptable salt thereof, for use in treatment or prevention of bacterial biofilms and/or other microbial infections 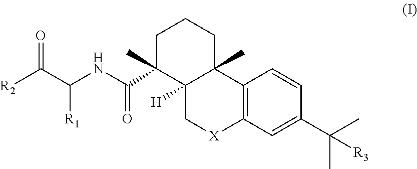
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl.

2. A compound of formula (I) 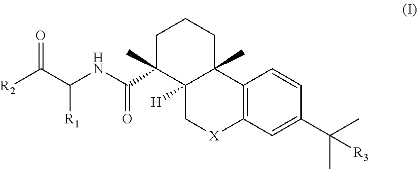
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₈-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₈-alkyl;
or a pharmaceutically acceptable salt thereof;
provided that when X is CH₂, R2 is OH, and R3 is H, R1 is not H, iso-propyl or benzyl, or when X is CH₂, R2 is OH, R3 is H, and R1 is in D-configuration, R1 is not isobutyl, p-OH substituted benzyl, indolyl or methyl-S-propanyl.

3. A compound of formula (I) 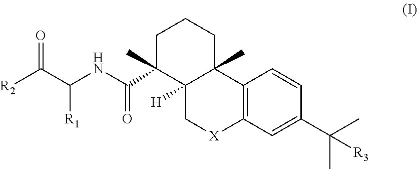
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl;
or a pharmaceutically acceptable salt thereof;
provided that
when X is CH₂, R2 is OH, and R3 is H, R1 is not H, Me, L—CH(CH₃)₂, CH₂OH, L'CH₂Ph, L-indolyl, L—(CH₂)COOH; L—(CH₂)₂COOH; (CH₂)₂SMe; or
when X is CH₂, R2 is OMe, and R3 is H, R1 is not H, L—Me, L—CH₂COOMe, L—CH(CH₃)CH₂CH₃, L—CH₂CH(CH₃)₂, CH₂Ph, L—CH₂OH, L—CH₂(C₆H₄)-p-OH or L—CH(CH3)₂.

4. The compound according to claim 2 or 3 for use as a medicament.

5. The compound according to claim 2 or 3 for use in treatment or prevention of bacterial biofilms and/or other microbial infections.

6. A compound according to claim 1, 2 or 3 for use in treatment or prevention of disorders caused by microbial growth and viability as well as bacterial colonization in a subject.

7. A compound according to claim 1, 2 or 3 for use in treatment or prevention of a disease or a condition involving or resulting from bacterial biofilms and/or other microbial infections.

8. A compound of formula (I), 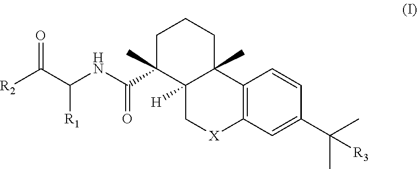
wherein
X is selected from CH₂ and C═O;
R1 is CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁-₃⁻(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH; and
R3 is H, OOH, COOR′, or OH;
wherein
R is H or C₁₋₃-alkyl; and
R′ is C₁₋₆-alkyl;
or a pharmaceutically acceptable salt thereof,
for use in treatment or prevention of a disease or a condition involving or resulting from bacterial biofilms and/or other microbial infections or treatment or prevention of disorders caused by microbial growth and viability as well as bacterial colonization in a subject.

9. A compound for use according to any one of claims 6 to 8 wherein treatment or prevention of a disease or a condition is reached by achieving a level of antibacterial or antimicrobial activity sufficient to inhibit bacteria or microbes, or the growth, viability or colonization thereof.

10. The compound for use according to any one of claims 1 and 4 to 9, or the compound according to claim 2 or 3, wherein R1 is CH₂—Cy, wherein Cy is C₃₋₈-cycloalkyl or a mono or bicyclic heterocyclyl or (hetero)aryl, optionally comprising 1 to 3 heteroatoms each independently selected from S, N and 0, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; in particular Cy is cyclohexyl, phenyl, pyridynyl, or indolyl, any of which may be optionally substituted as indicated.

11. The compound for use according to any one of claims 1 and 4 to 9, or the compound according to claim 2 or 3, wherein R1 is selected from the group consisting of —H, —CH(CH₃)₂, —CH₂CH₃, CH₂CH(CH₃)₂, CH₂CH₂SCH₃,

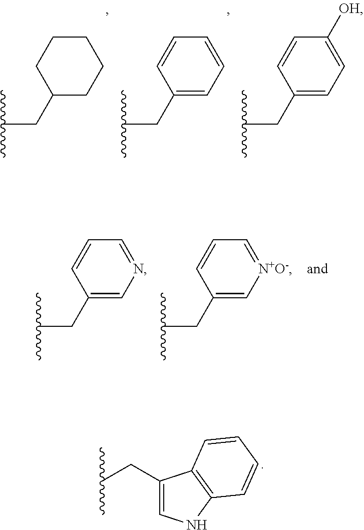

12. The compound for use according to any one of claims 1, and 4 to 11, or the compound according to any one of claims 2, 3 and 10 to 11, wherein X is CH₂ or C═O, preferably CH₂.

13. The compound for use according to any one of claims 1, and 4 to 12, or the compound according to any one of claims 2, 3 and 10 to 13, wherein R2 is OH or OR′.

14. The compound for use according to any one of claims 1, and 4 to 12, or the compound according to any one of claims 2, 3 and 10 to 13, wherein R2 is Y1.

15. The compound for use according to any one of claims 1, and 4 to 12, or the compound according to any one of claims 2, 3 and 10 to 13, wherein R2 is Y1Y2.

16. The compound for use according to any one of claims 1, and 4 to 15, or the compound according to any one of claims 2, 3 and 10 to 15, wherein Y1 and Y2 are each, when present, selected from histidine, alanine, isoleucine, arginine, leucine, asparagine, lysine, aspartic acid, methionine, cysteine, phenylalanine, cyclohexylalanine, glutamic acid, threonine, glutamine, tryptophan, glycine, valine, ornithine, serine and tyrosine; preferably from glycine, valine, leucine, phenylalanine, cyclohexylalanine, methionine, tyrosine, and tryptophane. 17 A method of coating a surface of a material, wherein said method comprises applying a composition comprising a compound of formula (I) 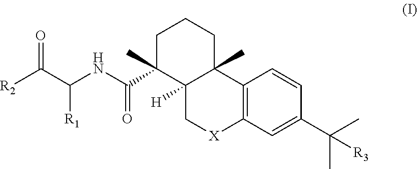
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₈-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₈-alkyl,
to the surface of the material.

18. The method of claim 17, wherein in the composition further comprises at least one other agents selected from the group consisting of solvent, diluent, carrier, buffer, excipient, adjuvant, antiseptic, and a filling, stabilizing, thickening, wetting, dispersing, solubilizing, suspending, emulsifying, binding, disintegrating, encapsulating, coating, embedding, lubricating, colouring, flavouring agent, absorbent, absorption enhancer, humectant, and preservative.

19. Use of a compound of formula (I) 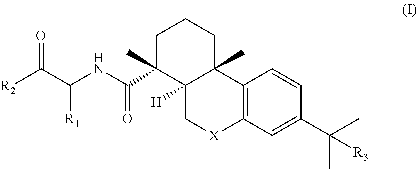
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₈-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₈-alkyl.
for coating a surface of a material.

20. A coating comprising a compound of formula (I) 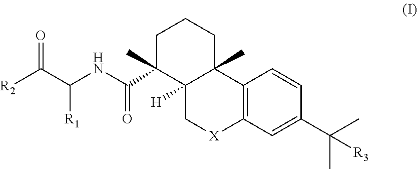
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and
wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl.

21. A surface coated material, wherein the coating comprises the compound of formula (I) 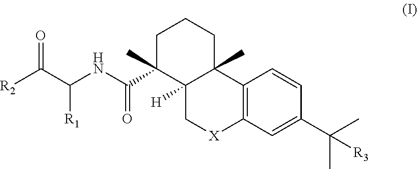
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl.

22. A method of preventing, reducing or inhibiting bacterial biofilm or microbial formation, wherein said method comprises applying a composition comprising the compound of formula (I) 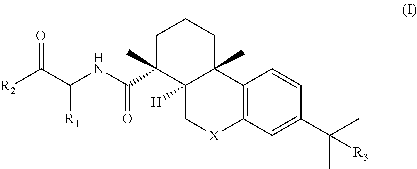
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl,
into a material or to a surface of a material.

23. Use of the compound of formula (I) 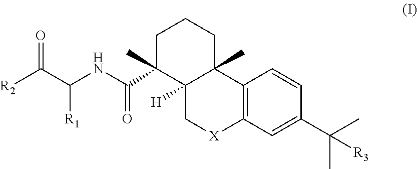
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₈-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₈-alkyl.
for preventing, reducing or inhibiting bacterial biofilm or microbial formation in or on a material.

24. Use of the compound of formula (I) 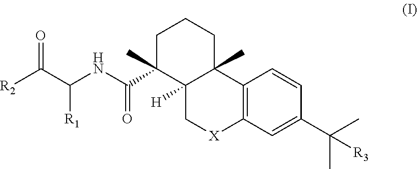
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₈-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₈-alkyl.
in medical devices, water filtration systems, ship hulls, textiles, furniture, food and food-related related surfaces, pharmaceuticals and devices for drug delivery, dressings, coatings, laboratory devices, biosensors, materials for patterned cell culture, diagnostic kits, cleaning solutions or desinfectants.

25. A method of treating or preventing disorders caused by microbial growth and viability as well as bacterial colonization in a subject, wherein said method comprises administering an effective amount of a composition comprising a compound of formula (I) 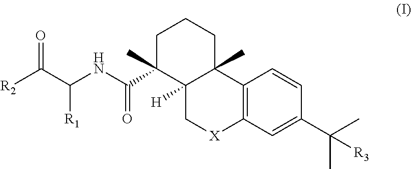
wherein
X is selected from CH₂, C═O and C═N—OH;
each R1 is independently selected from a group consisting of H; optionally substituted unbranched or branched, cyclic or acyclic C₁₋₈-alkyl, wherein the carbon chain is optionally interrupted once with NH, O or S; and CH₂—Cy, wherein Cy is C₃₋₈-cycloalkyl or a mono or bicyclic heterocyclyl or (hetero)aryl, optionally comprising 1 to 3 heteroatoms each independently selected from S, N and O, any of which may be optionally substituted one or more times; and wherein said optional substituents of R1 are each independently selected from the group consisting of halogen, C₁₋₃-alkyl, C₁₋₃-(per)haloalkyl, OR, SR, CN, NO₂, NHC(NH₂)₂, COR, COOR, CONHR, NR₂, NHCSR, NHCOR, NHCONHR, NHCOOR, OCOR, and OCONHR; and
R2 is OH, OR′ or an amino acid residue of formula —Y1 or a dipeptide residue of formula —Y1Y2 or a C₁₋₆-alkyl ester of said amino acid or said dipeptide residue; and
R3 is H, OOH, COOR′, or OH;
wherein
Y1 and Y2 are each independently selected from natural and non-natural amino acids comprising in its side chain 0 to 15 carbon atoms and optionally 1 to 4 heteroatoms;
R is H or C₁₋₃-alkyl; and
R′ is C₁₋₆-alkyl,
to the subject in need thereof.

26. A method according to any one of claims 17, 18, 22 and 25, a use according to any one of claim 19, 23, or 24, a coating according to claim 20, or a surface coated material according to claim 21, wherein R1 is selected from the group consisting of —H, —CH(CH₃)₂, —CH₂CH₃, CH₂CH(CH₃)₂, CH₂CH₂SCH₃,

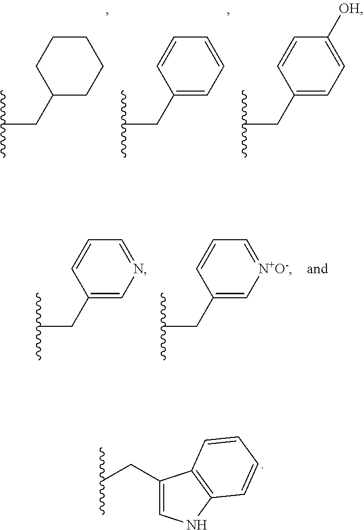

27. A method according to any one of claims 17, 18, 22, 25 and 26, a use according to any one of claims 19, 23, 24, and 26, a coating according to claims 20 and 26, or a surface coated material according to claims 21 and 26, wherein X is CH₂ or C═O, preferably CH₂.

28. A method according to any one of claims 17, 18, 22, 25 and 26, a use according to any one of claims 19, 23, 24, 26 and 27, a coating according to claims 20, 26 and 27, or a surface coated material according to claims 21 26 and 27, wherein R2 is OH or OR′, preferably OH.

29. The compound for use, use or method according to any one of the previous claims, wherein growth, viability or colonization of bacteria is inhibited or reduced.

30. The compound for use, use or method according to any one of the previous claims, wherein said bacteria are Gram-positive bacteria, Gram-negative bacteria, planktonic bacteria, bacteria growing in a biofilm or any combination thereof.

31. The compound for use, use or method according to claim 21, wherein said bacteria are selected from the group consisting of various strains of planktonic bacteria, Staphylococcus spp. including Staphylococcus aureus and Staphylococcus epidermidis, and Escherichia coli or any combination thereof.

32. The compound for use, use or method according to any one of the previous claims, wherein said disorder caused by bacteria is selected from the group consisting of bacterial infections, inflammation caused by bacteria, bacterial tissue damage, impetigo, lung pneumonia of cystic fibrosis patients, otitis media, chronic wounds, Legionnaire's disease, nosocomial infections and hospital-acquired infections.

33. The compound for use, use or method according to any one of the previous claims, wherein a molar concentration of the compound of formula (I) is about 0.5-1000 μM.

34. The method according to any one of the previous claims, wherein the composition is applied or administered once or several times.

35. The method according to any one of the previous claims, wherein the composition is applied or administered before, after or concurrently with another antimicrobial agent.

36. A process for preparing a compound of formula (I) as defined in claim 2 or 3, wherein said method comprises coupling of an amino acid residue or a peptidic residue to dehydroabietic acid in order to obtain the said compound of formula (I).

37. The process according to claim 36, wherein the process is accomplished following any one of the following synthesis routes: [chemical structure]
